(19) United States
(12) Patent Application Publication
(10) Pub. No.: US 2013/0111619 A1
(43) Pub. Date: May 2, 2013

(54) HIGH BIOMASS MISCANTHUS VARIETIES

(71) Applicants: Erik J. Sacks, Lafayette, IN (US); Katrin Jakob, Alameda, CA (US); Neal I. Gutterson, Oakland, CA (US)

(72) Inventors: Erik J. Sacks, Lafayette, IN (US); Katrin Jakob, Alameda, CA (US); Neal I. Gutterson, Oakland, CA (US)

(73) Assignee: Mendel Biotechnology Inc., Hayward, CA (US)

(21) Appl. No.: 13/513,173

(22) PCT Filed: Dec. 22, 2010
(86) PCT No.: PCT/US10/61898

§ 371 Date: Nov. 15, 2012

Related U.S. Application Data
(60) Provisional application No. 61/289,043, filed on Dec. 22, 2009.

(51) Int. Cl. C12N 15/82

(52) U.S. Cl. CPC C12N 15/8242
USPC 800/260; 800/298

(57) ABSTRACT

The present invention provides varieties of fertile Miscanthus that have greater water deficit tolerance, greater vigor, greater cold tolerance, later flowering, and/or higher biomass, i.e., greater biomass than a control Miscanthus plant, and methods for producing and using the said Miscanthus varieties. These varieties may be used to produce cellulosic biofuels, or to produce inbred or hybrid Miscanthus plants. Plant cells, seeds and other plant parts are also described.

### CROSS-REFERENCE TO RELATED APPLICATIONS

This application claims priority to U.S. Provisional Application No. 61/289,043, filed Dec. 22, 2009, which is hereby incorporated by reference in its entirety.

### FIELD OF THE INVENTION

The present invention pertains to seed-propagated varieties or cultivars of Miscanthus and, more particularly, to high biomass-yielding Miscanthus varieties or cultivars.

### BACKGROUND OF THE INVENTION

The production of significant amounts of biomass from various plant species can be an effective means of capturing and storing solar energy that is cost-competitive with petroleum-based fuels in various markets. For example, the market for transportation fuel is considerable, directly or indirectly impacting a large segment of the global economy, and the conversion of cellulosic material into cellulosic ethanol or other biofuels can meet many transportation needs. Alternatively, cellulosic feedstocks can be used to produce electricity via direct combustion when, for example, the material is used for co-firing in coal power generating facilities. This practice is occurring with growing frequency in the United Kingdom and Europe.

Diverse plant species that have been suggested as producers of harvestable biomass for fuel production include alfalfa, poplar, pine, Eucalyptus, Leucaena, soy, safflower, sunflower, cotton, tobacco, rape, sugar beet, corn, wheat, rice, sorghum, barley, ryegrass, turf grass, bamboo, sugarcane, willow, switchgrass, and Miscanthus, among many others. Relative to the other species in this list, Miscanthus has some important advantages, including very high biomass yield, low chemical input requirements, and little annual agronomic input once this perennial species is established in the field. See, for example, Heaton et al. (2008) Global Change Biology 14:2000-2014 and Christian et al. (2008) Industrial Crops and Products 28:320-327; and Jones and Walsh (2001) Miscanthus for energy and fibre, Earthscan, 192 pages.

However, the production of biomass, including biomass from Miscanthus, is not without its challenges. For example, the only Miscanthus types that have to date been demonstrated to be cost effective for biomass production are sterile, triploid clones of Miscanthus×giganteus (M×g), also known as Giant Miscanthus, a hybrid species that include chromosomes from both M. sinensis (Msi) and M. sacchariflorus (Msa). Miscanthus×giganteus has been chosen as a candidate biomass crop since it incorporates desirable traits from its parent species, M. sinensis and M. sacchariflorus, and has yields generally higher than either parent, through interspecies heterosis. Neither of the parent species is easily deployed as a biomass crop, despite their fertility. For example, though Msi cultivars are generally fertile, these cultivars generally have lower yields than M×g. In the United States, M. sinensis cultivars are common garden ornamentals.

One variety of M×g that has been proposed for commercial biomass production is the M.×giganteus “Illinois” clone (“M×g ‘Illinois’ clone” of the species Miscanthus×giganteus Greef et Deu ex. Hodkinson et Renvoize; Heaton et al. (2008a) Curr. Opin. Biotechnol. 19: 202-209, hereby incorporated by reference in its entirety; Heaton et al. (2008b) Global Change Biol. 14: 2000-2014, hereby incorporated by reference in its entirety), with which it has been suggested that 260% more ethanol per unit land area can be produced than is produced from corn grain (Heaton et al., 2008a, 2008b, supra). Sterile clones of M×g must be propagated asexually either with cuttings or rhizomes, which are directly planted in the field to establish new plantations of this biomass crop. Compared to planting with seed from fertile varieties, establishing a plantation from vegetative material is costly and tends to limit planting density, which in turn limits the ability to generate a harvestable crop after the first growing season, or to produce commercially desirable yields in the second growing season.

Miscanthus reproductive biology limits one's options for production of planting materials with desirable commercial characteristics. Most Miscanthus species are self-incompatible, meaning that they have conditional fertility. When most Miscanthus lines are grown in isolation, away from other Miscanthus lines, no or only a very few seeds are produced, as the pollen of that plant cannot fertilize that plant since the pollen and egg cells are of the same compatibility group. However, when two Miscanthus lines with different incompatibility groups are grown adjacently, each line can produce pollen capable of fertilizing the other line. Thus, most Miscanthus lines are capable of producing hundreds-fold more seed when grown near a line with a different compatibility group than when grown in isolation.

Fertile tetraploid Miscanthus lines can be generated by several means, including breeding. Triploid and tetraploid Miscanthus progeny resulting from crossing diploid M. sinensis with tetraploid M. sacchariflorus have been reported by Hirayoshi et al. (1960) Res. Bull. Fac. Agr. Gifu Univ. 12: 82-88, and by Matumura et al. (1985) Res. Bull. Fac. Agr. Gifu Univ. 50: 423-433; Matumura et al. (1986) Res. Bull. Fac. Agr. Gifu Univ. 51: 347-362; Matumura et al. (1987) Res. Bull. Fac. Agr. Gifu Univ. 52: 315-324. Matamura observed that yields of the 4× progeny were higher than the 3× progeny and both parents, about twice the yield of the sinensis parent and four times the yield of the sacchariflorus parent. Fertile tetraploid Miscanthus lines have also been generated through the polyploidization of Miscanthus sinensis and Miscanthus×giganteus with colchicine treatment (Glowacka et al. (2009) Indust. Crops Products 30: 444-449).

In addition to it uses as a high yielding biofuel feedstock, Miscanthus also has potential benefits for soil stabilization/improvement, water filtration, wildlife cover and carbon sequestration.

Substantial improvements to Miscanthus lines will be required through breeding to generate commercially viable biomass varieties. What are needed, therefore, are Miscanthus varieties that have the desirable yields resulting from the combination of the chromosomes of M. sinensis and M. sacchariflorus, but which can be propagated through seed, and which have other advantageous, such as those described herein with the presently described plants.

### SUMMARY OF THE INVENTION

The present invention is directed to varieties of high biomass-yielding, fertile tetraploid Miscanthus germplasm (“FTMG”), and methods for producing and using said Miscanthus varieties. These varieties are tetraploid, rather than diploid, and as a result retain fertility. Such varieties can be produced by crossing tetraploid M. sacchariflorus lines with diploid M. sinensis lines. The result of such crosses is most commonly sterile triploid clones; however, FTMG lines can be identified by screening for DNA content or chromosome number of progeny of a diploid Msi×tetraploid Msa, and then testing such clones for fertility. Alternatively, clones can be tested for fertility, for example, by growing near other fertile, incompatible Miscanthus lines, and then chromosome number of DNA content can be measured.

The present invention also pertains to fertile, tetraploid FTMG varieties that produce biomass yield similar to or greater than the sterile, triploid M×g clones currently used for biomass production, such as, for example, a control plant of M×g ‘Illinois’ clone (Heaton et al. (2008a, 2008b) supra). The average biomass yield of the FTMG varieties will generally be at least 75%, at least 80%, at least 85%, at least 90%, at least 95%, at least 100%, at least 105%, at least 110%, at least 115%, at least 120%, or at least 125%, or more, of the biomass produced by a control Miscanthus×giganteus variety, such as, for example, the M×g ‘Illinois’ clone. The latter, which is known to produce a desirable biomass yield under appropriate environmental conditions, is sterile and unable to produce seed. The fertile, tetraploid varieties that are the subject of the instant invention may be selected for having yield similar to an M×g sterile triploid control plant (for example, the M×g ‘Illinois’ clone), when the fertile varieties and control plants are at substantially the same stage of seedling development having been grown under substantially the same environmental conditions. The invention is also directed to a plant cell, a plant part, a tissue culture of regenerable cells, or a seed of the fertile Miscanthus varieties.

Seed of these fertile, tetraploid FTMG varieties may be used to establish Miscanthus plantations for the production of feedstock for cellulosic biofuel conversion facilities or electricity generation facilities. These fertile tetraploid FTMG can also produce inbred or hybrid Miscanthus plants. Plant cells, seeds and other plant parts derived from plants grown from these seed are also described.

Seed for commercially effective establishment of Miscanthus plantations can be produced in a number of ways. Since individual lines of fertile, tetraploid FTMG can be propagated clonally and are generally self-incompatible, seed production fields can be established with two or more genetically distinct lines that are cross-compatible to produce seed cost-effectively (Syn1 seed). Syn1 seed can be harvested annually from these fields to produce seed with highly reproducible characteristics on a plantation scale. Syn1 seed collected from these fields can also be used to establish seed production fields. In this case, the seed from these production fields are Syn2 seed, and the resulting plants produced from Syn2 seed have similar characteristics as plants derived from Syn1 seed, but less so than for successive lots of Syn1 seed. This process can be repeated, yielding Syn 3, Syn4, etc. seed.

Any of the plants grown from the Syn1, Syn2, etc. seed are each fertile, tetraploid FTMG clones, which can be used as parental lines that can be propagated for seed production as described above. These parental lines can be selected for further desirable features, for example, altered flowering time, improved biomass yield, increased water deficit tolerance, increased water deficit tolerance, etc., to produce further improved varieties of fertile, tetraploid FTMG.

Genetic improvement of fertile tetraploid FTMG can be achieved by crossing fertile tetraploid FTMG lines with other fertile tetraploid FTMG lines. Genetic improvement of fertile tetraploid FTMG lines can also be achieved by crossing with tetraploid M. sinensis or M. sacchariflorus lines that have desirable features. Such tetraploid M. sinensis or M. sacchariflorus are generally produced by doubling the chromosome number of desirable diploid lines of Msi or Msa, but tetraploid lines may be found in nature (e.g., M. sacchariflorus varieties found in Japan).

The present invention is also directed to fertile tetraploid Miscanthus varieties which have an average stem diameter similar to or greater than the stem diameter of a sterile, triploid M×g clones, such as, for example, a control plant of Miscanthus×giganteus ‘Illinois’ when grown under substantially the same environmental conditions. The average stem diameter of the FTMG varieties will generally be at least 75%, at least 80%, at least 85%, at least 90%, at least 95%, at least 100%, at least 105%, at least 110%, at least 115%, at least 120%, or at least 125%, or more, of the stem diameter of a control Miscanthus×giganteus variety, such as, for example, the M×g ‘Illinois’ clone.

The present invention is also directed to fertile tetraploid Miscanthus varieties which produce biomass yield similar to or greater than the sterile, triploid M×g clones currently used for biomass production, such as, for example, a control plant of M×g ‘Illinois’ clone, and have an average stem diameter similar to or greater than the stem diameter of a sterile, triploid M×g clones, such as, for example, a control plant of Miscanthus×giganteus ‘Illinois’ when grown under substantially the same environmental conditions. The average biomass yield of the FTMG varieties will generally be at least 75%, at least 80%, at least 85%, at least 90%, at least 95%, at least 100%, at least 105%, at least 110%, at least 115%, at least 120%, or at least 125%, or more, of the biomass produced by a control Miscanthus×giganteus variety, such as, for example, the M×g ‘Illinois’ clone. The average stem diameter of the FTMG varieties will generally be at least 75%, at least 80%, at least 85%, at least 90%, at least 95%, at least 100%, at least 105%, at least 110%, at least 115%, at least 120%, or at least 125%, or more, of the stem diameter of a control Miscanthus×giganteus variety, such as, for example, the M×g ‘Illinois’ clone.

In some embodiments, the fertile tetraploid Miscanthus varieties of the present invention produce a biomass yield at least 100% of the biomass yield produced by Miscanthus×giganteus ‘Illinois.’ In some embodiments, the fertile tetraploid Miscanthus varieties of the present invention produce a biomass yield at least 105% of the biomass yield produced by Miscanthus×giganteus ‘Illinois.’ In some embodiments, the fertile tetraploid Miscanthus varieties of the present invention produce an average stem diameter at least 100% of the average stem diameter of Miscanthus×giganteus ‘Illinois.’ In some embodiments, the fertile tetraploid Miscanthus variety of the present invention produce an average stem diameter at least 105% of the average stem diameter of Miscanthus×giganteus ‘Illinois.’

In some embodiments, the fertile tetraploid Miscanthus varieties of the present invention comprise germplasm which traces its origin to one or more varieties selected from the group consisting of ‘MBS 7002,’ ‘MBS 7003,’ ‘MBS 1001,’ and ‘MBS 1002.’

The present invention also provides Miscanthus hybrid, synthetic or open pollinated populations wherein at least one parent used to produce said hybrid, synthetic or open pollinated populations is selected from the group of Miscanthus varieties consisting of ‘MBS 7002,’ ‘MBS 7003,’ ‘MBS 1001,’ and ‘MBS 1002.’

The present invention also provides Miscanthus hybrid, synthetic or open pollinated populations wherein said hybrid, synthetic or open pollinated populations comprise germplasm from one or more Miscanthus varieties selected from the group of varieties consisting of ‘MBS 7002,’ ‘MBS 7003,’ ‘MBS 1001,’ and ‘MBS 1002.’

In some embodiments, the Miscanthus hybrid, synthetic or open pollinated populations of the present invention comprise fertile tetraploid Miscanthus plants.

The present invention also provides Miscanthus hybrids. In some embodiments, the hybrids are selected from the group consisting of ‘MBS 7002’×‘MBS 7003’; ‘MBS 7002’×‘MBS 1001’; ‘MBS 7002’×“MBS 1002’; ‘MBS 7003’×‘MBS 1001’; ‘MBS 7003’×‘MBS 1002’; and ‘MBS 1001’×‘MBS 1002.’

The present invention also provides Miscanthus hybrid, synthetic or open pollinated populations. In some embodiments, the Miscanthus hybrid, synthetic or open pollinated populations are selected from the group consisting of ‘MBS 7002’×‘MBS 7003’×‘MBS 1001’; ‘MBS 7002’×‘MBS 7003’×‘MBS 1002’; ‘MBS 7002’×‘MBS 1001’×‘MBS 1002’ ‘MBS 7003’×‘MBS 1001’×‘MBS 1002’, and ‘MBS 7002’×‘MBS 7003’×‘MBS 1001’×‘MBS 1002.’

The present invention further relates to methods of producing Miscanthus hybrid, synthetic or open pollinated populations. In some embodiments, the methods comprise crossing two or more fertile tetraploid Miscanthus varieties wherein at least one parent used to produce said hybrid, synthetic or open pollinated population is selected from the group of Miscanthus varieties consisting of ‘MBS 7002,’ ‘MBS 7003,’ ‘MBS 1001,’ and ‘MBS 1002.’ In some embodiments, at least two parents used to produce said hybrid, synthetic or open pollinated population are selected from the group of Miscanthus varieties consisting of ‘MBS 7002,’ ‘MBS 7003,’ ‘MBS 1001,’ and ‘MBS 1002.’ In some embodiments, at least three parents used to produce said hybrid, synthetic or open pollinated population are selected from the group of Miscanthus varieties consisting of ‘MBS 7002,’ ‘MBS 7003,’ ‘MBS 1001,’ and ‘MBS 1002.’. In some embodiments, the parents used to produce said hybrid, synthetic or open pollinated population comprise ‘MBS 7002,’ ‘MBS 7003,’ ‘MBS 1001,’ and ‘MBS 1002.’

The present invention also relates to methods of biofuel production. In some embodiments, the methods comprise using feedstock for said biofuel production, wherein said feedstock comprises plant biomass produced by a Miscanthus variety of the present invention. In some embodiments, the feedstock is selected from the group consisting of ‘MBS 7002,’ ‘MBS 7003,’ ‘MBS 1001,’ ‘MBS 1002,’ or combination thereof.

The present invention is also directed to a method of imparting an altered trait to a plant such as a Miscanthus plant, as compared to a control plant, and the altered trait includes producing a similar or greater biomass yield (generally, this is at least 75%, at least 80%, or at least 85%, at least 90%, at least 95%, at least 100%, at least 105%, or at least 110%, at least 115%, at least 120%, at least 125% or more of the yield of biomass produced by sterile, triploid M×g, for example the M×g ‘Illinois’ clone), greater tolerance to water deficit that the tolerance of sterile, triploid M×g, for example, the ‘Illinois’ clone, or another control plant, greater cold tolerance than M. sinensis or another control plant, etc. The method steps include crossing a first Miscanthus plant that produces similar or greater yield to the control plant, with a second Miscanthus plant that has more tolerance to water deficit or greater seedling vigor than the first plant or a control plant, or with a second Miscanthus plant that has more tolerance to cold than the first plant or a control plant (e.g., M. sinensis), particularly when the experimental and control plants are at the seedling stage. A suitable control plant may include a Miscanthus variety such as, for example, the M×g ‘Illinois’ clone (Heaton et al. (2008a, 2008b, supra), or a parental line. Optionally, the method further comprises a screening process for identifying the altered trait in the plant.

The present invention is also directed to a method of introducing a heritable trait into a Miscanthus plant, wherein the heritable trait is at least similar biomass yield, later flowering, increased seedling vigor, increased cold tolerance, increased disease resistance, or greater tolerance to water deficit than a control plant, wherein the control plant may be, for example, the M×g ‘Illinois’ clone, or in the case of cold tolerance or seedling vigor, a variety of M. sinensis. The steps of this method include

(a) crossing a Miscanthus plant with another Miscanthus plant that heritably carries the heritable trait (for example, Miscanthus varieties ‘MBS 7002,’ ‘MBS 7003,’ ‘MBS 1001,’ or ‘MBS 1002’) to produce F₁ progeny plants, at least some of which heritably carry the trait;

(b) selecting F₁ progeny plants that heritably carry the trait;

(c) crossing the selected progeny plants with another plant (for example, a Miscanthus plant or any other cross-compatible genus) to produce next-generation progeny plants at least some of which heritably carry the trait;

(d) selecting next-generation progeny plants that heritably carry the heritable trait; and optionally

(e) repeating steps (c) and (d) to produce selected progeny plants that comprise the heritable trait.

The present invention also pertains to the use of a Miscanthus seed to produce a Miscanthus variety having cold tolerance, greater seedling vigor, greater water deficit tolerance and/or at least similar biomass yield compared to a control plant (that is, at least 75% to 125% or more of the biomass yield of the control plant), said seed produced by crossing (in either direction) an FTMG plant having cold tolerance, greater seedling vigor, greater water deficit tolerance with a second Miscanthus plant having at the least similar biomass yield as compared to the control plant (that is, a biomass yield of at least 75%, at least 80%, at least 85%, at least 90%, at least 95%, at least 100%, at least 105%, at least 110%, at least 115%, at least 120%, at least 125% or more of the biomass yield of the control plant). Again, a suitable control plant that can be used for comparison purposes for yield, water deficit tolerance or vigor can be the M×g ‘Illinois’ clone, and for cold tolerance or seedling vigor, a suitable control plant may include a variety of M. sinensis.

The present invention is also directed to a population of fertile, tetraploid Miscanthus plants, such as a population of crop plants in the field. Because the present invention provides several genetically distinct FTMG varieties any of which, or progeny plants derived from crosses of these FTMG varieties, may be valuable for biomass production, the advantages of genetic diversity in this crop become apparent to the skilled artisan or grower. A genetically diverse crop is likely to be more resistant to diseases and pests than a crop that may be produced with a single variety or plant line, such as, for example, the M×g ‘Illinois’ clone, or a single plant variety taught in the scientific literature, which has been touted as an interesting candidate biomass-producing crop or a fertile variety of unknown yield potential.

The presently described FTMG varieties may also be used in a novel method to produce high-biomass Miscanthus progeny plants from seed. The first step in this method includes crossing a first fertile tetraploid high-biomass yielding Miscanthus plant (e.g., one of the FTMG varieties) with a second fertile tetraploid high-biomass yielding Miscanthus plant (a different FTMG variety). The seeds that result from the crossing may then be harvested and grown to produce the high-biomass progeny Miscanthus plant. Optionally, the high-biomass progeny Miscanthus plant may be selected from a plurality of plants produced by this method on the basis of biomass yield and possibly other properties (e.g., seedling vigor, water deficit tolerance). The high biomass value of the plants produced by this method may be evaluated by comparison to a standard, such as a particular percentage of the yield of the biomass produced by the M×g ‘Illinois’ clone when the progeny Miscanthus plant, the first fertile tetraploid high-biomass Miscanthus plant, the second fertile tetraploid high-biomass Miscanthus plant, or the M×g ‘Illinois’ clone are harvested at substantially the same stage of development having been grown under substantially the same environmental conditions.

### BRIEF DESCRIPTION OF THE DRAWINGS

FIG. 1 provides a schematic of the breeding methodology used to create the Miscanthus varieties ‘MBS 7001’ (i.e., the 3× sterile ‘Nagara’), ‘MBS 7002,’ ‘MBS 7003,’ ‘MBS 1001’ and ‘MBS 1002’ (left-hand side); and the process of intermating (i.e., crossing) ‘MBS 7002,’ ‘MBS 7003,’ ‘MBS 1001’ and ‘MBS 1002’ in various ways so as to create all possible two, three and four combination crosses between and among these varieties (right-hand side).

FIG. 2 provides a schematic of the breeding methodology used to create the Miscanthus varieties ‘MBS 7001’ (i.e., the 3× sterile ‘Nagara’), 00m0007002 (aka ‘MBS 7002’ or ‘Lake Erie’), 00 m000703 (aka ‘MBS 7003’ or ‘Columbia’), 00 m0007004 (aka ‘MBS 1001’) and 00 m0007005 (aka ‘MBS 1002’) (top half); and the process of intermating (i.e., crossing) ‘MBS 7002,’ ‘MBS 7003,’ ‘MBS 1001’ and ‘MBS 1002’ to create fertile tetraploid polycross sibs (bottom half). “MBS 7002♀” means the sibs designated as 07s0031 are created by using MBS 7002 as the female parent; “MBS 7003♀” means the sibs designated as 07s0032 are created by using MBS 7003 as the female parent; “MBS 7004♀” means the sibs designated as 07s0033 are created by using MBS 7004 as the female parent; and “MBS 7005♀” means the sibs designated as 07s0034 are created by using MBS 7002 as the female parent.

### DETAILED DESCRIPTION OF THE INVENTION

It will be readily apparent to the skilled artisan that various substitutions and modifications may be made in the invention disclosed herein without departing from the scope and spirit of the invention.

It is noted that as used herein, the singular forms “a,” “an,” and “the” include plural reference unless the context clearly dictates otherwise. Thus, for example, a reference to “a plant” or “a variety” includes one or a plurality of such plants or varieties, and a reference to “a stress” is a reference to one or more stresses and equivalents thereof known to those skilled in the art, and so forth.

### DEFINITIONS

The term “plant” includes whole plants, shoot vegetative organs/structures (for example, leaves, stems and tubers), roots, flowers and floral organs/structures (for example, bracts, sepals, petals, stamens, carpels, anthers and ovules), seed (including embryo, endosperm, and seed coat) and fruit (the mature ovary), plant tissue (for example, vascular tissue, ground tissue, and the like) and cells (for example, guard cells, egg cells, and the like), and progeny of same. The class of plants that can be used in the method of the invention is generally as broad as the genus of Miscanthus, or may be applied more narrowly to Miscanthus species, subspecies cultivars, varieties, and/or hybrids.

A “control plant” as used in the present invention refers to a plant cell, seed, plant component, plant tissue, plant organ or whole plant used to compare against an instant Miscanthus plant for the purpose of identifying an enhanced phenotype in the instant plant. A control plant may in some cases be a parental Miscanthus plant line, or a species, subspecies, cultivar, variety, or hybrid that is an often-used or recognizable variety, for example, Miscanthus×giganteus, or more specifically, the M×g ‘Illinois’ clone. In other cases, a parental species may be used a control, including, but not limited to, M. sinensis varieties.

A “trait” refers to a physiological, morphological, biochemical, or physical characteristic of a plant or particular plant material or cell. In some instances, this characteristic is visible to the human eye, such as seed or plant size or seedling vigor, or can be measured by biochemical techniques, or by observation of a metabolic or physiological process, e.g. by measuring tolerance to water deprivation or cold, or by the observation of the expression level of a gene or genes, e.g., by employing Northern analysis, RT-PCR, microarray gene expression assays, or reporter gene expression systems, or by agricultural observations such as water deficit tolerance, low nutrient tolerance, hyperosmotic stress tolerance, cold tolerance or biomass yield. Any technique can be used to measure the amount of, comparative level of, or difference in the instant and control plants, however.

When two or more plants have “similar morphologies”, “substantially similar morphologies”, “a morphology that is substantially similar”, or are “morphologically similar”, the plants have comparable forms or appearances, including analogous features such as overall dimensions, height, width, mass, root mass, shape, glossiness, color, stem diameter, leaf size, leaf dimension, leaf density, internode distance, branching, root branching, number and form of inflorescences, and other macroscopic characteristics, and the individual plants are not readily distinguishable based on morphological characteristics alone.

When two or more plants are “at substantially the same stage of development”, they are at or very nearly at similar stages in their growth cycles, that is, having gone through cell division, cell enlargement, followed by cell differentiation and organ development to the same or very nearly the same degree, or they are in substantially the same stage of a specific phase of the life cycle such as an emergence phase, vegetative phase, reproductive phase or senescent phase.

When two or more plants are grown “under substantially the same environmental conditions”, they are grown in the same or very nearly the same temperatures, atmospheres (including carbon dioxide and oxygen concentrations), radiation wavelengths and flux, humidity, pathogen exposure, pest exposure, soil or growth medium quality, including pH, microflora, porosity, adsorption, absorption, nutrient or moisture levels, chemical growth enhancer levels, herbicide or pesticide levels, and to the same or very nearly the same quality, quantity and degree of the many other variables that may affect the plants' growth and development.

“Yield” or “plant yield” refers to increased plant growth, increased crop growth, increased biomass, and/or increased plant product production, and is dependent to some extent on temperature, plant size, organ size, planting density, light, water and nutrient availability, and how the plant copes with various stresses, such as through temperature acclimation and water or nutrient use efficiency. For example, Miscanthus has been reported to provide a yield of up to 18-20 tonnes of dry matter per hectare per year in one trial in Germany, but with significant variation in dry matter yield between sites in the first four years after planting (Jones and Walsh, ed. (2001) Miscanthus for Energy and Fibre, James & James, London, at page 62). Harvestable yields of Miscanthus in Europe have been reported to range from 10 to 40 tonnes of dry matter per hectare per year (Lewandowski et al, (2000) Biomass and Bioenergy 19: 209-227; Heaton et al. 2008b. supra). Heaton et al. have reported that fully established plants Miscanthus can provide typical autumn yields of dry matter ranging from 10 to 30 tonnes per hectare per year, depending on local agronomic conditions (Heaton et al. (2004) Mitigation and Adaptation Strategies for Global Change 9: 433-451). Miscanthus×giganteus autumn yields in lowland areas in Europe are typically higher than 25 tonnes per hectare per year, and Miscanthus×giganteus could provide a hypothetical yield of 27-44 tonnes of dry matter per hectare per year with a mean yield of 33 tonnes of dry matter per hectare per year in ‘Illinois’ (Heaton et al. (2004) supra). Miscanthus×giganteus can thus yield, under various conditions of growth, biomass of at least 10, at least 15, at least 20, at least 25, at least 27, at least 30, at least 33, at least 35, at least 40, at least 44 tonnes or more of dry matter per hectare per year. It is expected that the fertile, tetraploid varieties of Miscanthus (FTMG) described herein can produce similar biomass yields, ranging from, for example, at least 75%, at least 80%, at least 85%, at least 90%, at least 95%, at least 100%, at least 105%, at least 110%, at least 115%, at least 120%, at least 125% or more of the biomass yield of a control sterile triploid M×g crop at substantially the same stage of seedling development and grown under substantially the same, or the same, environmental conditions as the FTMG varieties, or, in other words, FTMG varieties are expected to yield at least 75% to at least 125% or more of 10 to 44 tonnes or more of dry matter per hectare per year.

“Planting density” refers to the number of plants that can be grown per acre. For crop species, planting or population density varies from a crop to a crop, from one growing region to another, and from year to year. Using corn as an example, the average prevailing density in 2000 was in the range of 20,000-25,000 plants per acre in Missouri, USA. A desirable higher population density (a measure of yield) would be at least 22,000 plants per acre, and a more desirable higher population density would be at least 28,000 plants per acre, more preferably at least 34,000 plants per acre, and most preferably at least 40,000 plants per acre. The average prevailing densities per acre of a few other examples of crop plants in the USA in the year 2000 were: wheat 1,000,000-1,500,000; rice 650,000-900,000; soybean 150,000-200,000, canola 260,000-350,000, sunflower 17,000-23,000 and cotton 28,000-55,000 plants per acre (Cheikh et al. (2003) U.S. Patent Application No. 20030101479). For Miscanthus, a typical initial planting density is 10,000 plants per hectare (Scurlock (1999) Miscanthus: A Review of European Experience with a Novel Energy Crop, U.S. Department of Energy, Publ. ORNL/TM-13732, at page 6). A desirable higher population density for each of these examples, as well as other valuable species of plants, including Miscanthus, would be at least 5%, or at least 10%, or at least 15%, or at least 20%, or at least 25%, or higher, than the average prevailing density or yield.

Plant breeders have historically used a various breeding, hybridization and selection techniques to create improved plant types. “Population improvement” can be used for the improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes for flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure. The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

In “mass selection,” desirable individual plants are chosen, harvested, and the seed composited without progeny testing to produce the following generation. Since selection is based on the maternal parent only, and there is no control over pollination, mass selection amounts to a form of random mating with selection. As stated above, the purpose of mass selection is to increase the proportion of superior genotypes in the population.

A “synthetic” variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and may thus be considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

A “hybrid” is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

Commercial Miscanthus seed may be provided either in a synthetic variety or a hybrid variety. Commercial production of synthetic varieties may include a breeder seed production stage, a foundation seed production stage, a registered seed production stage and a certified seed production stage. Hybrid variety seed production may involve up to three stages including a breeder seed production stage, a foundation seed production stage and a certified seed production stage.

The ability to produce and plant seed of biomass-yielding species has significant practical and financial implications. For example, the cost and effort of seed generation is significantly less than that associated with seedlings or plugs containing rhizomes, and can also result in improved volume and throughput. Sowing seed derived from Miscanthus species, for example, will generally cost less than the costs that would be associated with sowing plugs or seedlings. Farmers can thus plant more seeds with less cost, and with less effort, which allows for more plants to be seeded per unit area. The resulting initial higher planting density would bring about reduced costs per unit mass. As there is a significant positive correlation between initial planting density and yield in the first few years of growth (Jones and Walsh, ed., 2001, supra, at page 62), higher planting densities may also allow the farmer to produce for a commercially serviceable crop at the end of the first year of growth and better profit margins for the first few years after planting.

Miscanthus varieties have been developed through a combination of breeding and selection processes, the latter used to select for advantageous traits including, but not limited to, fertility, improved biomass, increased vigor, increased vigor at the seedling stage, increased water deficit tolerance, and greater tiller density. These improved characteristics were shown to be heritable, and it is expected that further improvements may be made with these varieties.

Specifically, the Miscanthus varieties ‘MBS 7002’(aka ‘Lake Erie’), ‘MBS 7003’(aka ‘Columbia’), ‘MBS 1001’ (aka ‘MBS 7004’), ‘MBS 1002’ (aka ‘MBS 7005’) were derived from interspecific crosses of Miscanthus sacchariflorus, a late flowering, highly rhizomatous, tetraploid species from Japan, and Miscanthus sinensis, an early flowering diploid species from China. After the crossing of the M. sacchariflorus and M. sinensis species, new varieties of high biomass yielding clones were selected, and at least four tetraploid varieties were selected that possessed advantageous properties over the parental species, as well as commercial varieties of Miscanthus giganteus. Each of these properties is likely to provide advantages to the grower. These advantageous properties include:
Greater water deficit tolerance of the ‘MBS 7002,’ ‘MBS 7003,’ ‘MBS 1001,’ ‘MBS 1002’ varieties than the M. sinensis parental lines and M.×giganteus variety ‘IL’. For example, the varieties ‘MBS 7002,’ ‘MBS 7003,’ ‘MBS 1001,’ ‘MBS 1002’ each demonstrated greater survival during a period of water deficit than the M. sinensis or M.×giganteus lines. Improved water deficit tolerance would increase the yield of Miscanthus in periods of reduced water availability, reduce the need for replanting, and increase the commercially viable range of this crop species.
Miscanthus varieties ‘MBS 7002,’ ‘MBS 7003,’ ‘MBS 1001,’ ‘MBS 1002’ had greater seedling vigor than the M. sinensis parental lines. Many weeds outgrow slow-growing young crops or out-compete them for nutrients, and thus it is usually desirable to use plants that establish themselves quickly. Seedlings and young plants are also particularly susceptible to stress conditions such as salinity or disease. Increasing seedling growth rate and shortening the time to emergence from soil contributes to seedling vigor, aids seedlings in coping with these stresses, and may allow these crops to be planted earlier in the season. Early planting helps add days to a growing season and may thus increase yield. Modification of the biomass of other tissues, such as root tissue, may be useful to improve a plant's ability to grow under harsh environmental conditions, including drought, high salt or nutrient deprivation, because larger roots may better reach or take up water or nutrients.
Miscanthus varieties ‘MBS 7002,’ ‘MBS 7003,’ ‘MBS 1001,’ ‘MBS 1002’ are generally later flowering than their M. sinensis parents, a characteristic that likely contributed to their greater height, and ultimately, may have contributed to their high biomass yield. Late flowering is generally useful in crops where the vegetative portion of the plant is the marketable portion; vegetative growth often stops when plants make the transition to flowering. Thus, it may be advantageous to prevent or delay flowering in order to increase yield of biomass. Prevention of flowering would also be useful in these same crops in order to prevent the spread of transgenic pollen and/or to prevent seed set.
Miscanthus varieties ‘MBS 7002,’ ‘MBS 7003,’ ‘MBS 1001,’ ‘MBS 1002’ have greater tiller density than the M. sacchariflorus parents, or M.×giganteus variety ‘IL’. Greater tiller density may result in high dry matter yield.
Significantly, and surprisingly, Miscanthus varieties ‘MBS 7002,’ ‘MBS 7003,’ ‘MBS 1001,’ ‘MBS 1002’ are tetraploid and fertile, as opposed to M.×giganteus, the latter being triploid and thus sterile. As discussed above, the cost and effort associated with seed production is significantly less than that associated with seedlings or plugs containing rhizomes. Farmers can thus sow more plants with less cost or effort, which allows for more plants to be seeded per unit area, possibly resulting in a commercially serviceable crop at the end of the first year of growth and higher yields for the first few years after planting.

The instant invention also relates to seeds derived from a fertile, high biomass yielding Miscanthus plant, for example, the plant of varieties ‘MBS 7002’(aka ‘Lake Erie’), ‘MBS 7003’(aka ‘Columbia’), ‘MBS 1001’ (aka ‘MBS 7004’), ‘MBS 1002’ (aka MBS 7005), descriptions of which are provided as follows. The following traits have been repeatedly observed and represent the characteristics of these cultivars. These cultivars have not been observed under all possible environmental conditions. The phenotype may vary somewhat with variations in temperature, day-length, light intensity, soil types, and water and fertility levels without, however, any variance in genotype.

Miscanthus varieties ‘MBS 7002,’ ‘MBS 7003,’ ‘MBS 1001’ and/or ‘MBS 1002’ are described in U.S. Provisional Patent Application No. 61/050,162, filed May 2, 2008; U.S. patent application Ser. No. 12/387,437 (filed May 1, 2009); U.S. patent application Ser. No. 12/387,429, filed May 1, 2009; and U.S. patent application Ser. No. 12/584,496, filed Sep. 4, 2009. Each and every one of these patent applications are hereby incorporated by reference in their entirety for all purposes. More specific information on these four varieties is provided in the following descriptions.

‘MBS 7002’ (aka ‘Lake Erie’).

The following traits have been repeatedly observed and represent the characteristics of the new cultivar. The new cultivar ‘MBS 7002’ has not been observed under all possible environmental conditions. The phenotype may vary somewhat with variations in temperature, day-length, light intensity, soil types, and water and fertility levels without, however, any variance in genotype.

The following traits have been repeatedly observed and are determined the basic characteristics of ‘MBS 7002’, which in combination distinguish this Miscanthus hybrid from the known Miscanthus×giganteus and other ornamental M. sinensis forms.

1. Vigorous growth

2. Top leaf height about 2.7 meters

3. Green leaves, no colored stripes are present

4. High biomass yield (about 20-30 tonnes per hectare)

5. High tiller density

‘MBS 7002’ can be distinguished from the Miscanthus cultivars ‘Strictus,’ ‘Super Stripe,’ ‘Gold Bar,’ ‘Little Zebra’ and ‘Mysterious Maiden’ in that ‘MBS 7002’ has no stripes or colored bands on its leaves.

In side by side comparisons conducted in Klein-Wanzleben, Germany, ‘MBS 7002’ is more vigorous than either of its parent plants and produces more biomass than either parent. ‘MBS 7002’ has taller culms but demonstrates less lodging; hence it has stronger culms. The leaves stay longer on the culm compared to M.×giganteus and, therefore, the leaf loss during the winter is minimized which, in turn, leads to higher biomass yield.

The plant can be propagated by rhizomes, from meristem or nodes. This further distinguishes ‘MBS 7002’ from M. sinensis in that M. sinensis cannot be propagated by nodes. “MBS 7002” develops inflorescences and viable seeds under optimal growing conditions.

The following observations, measurements, and comparison describe this plant as grown at Klein-Wanzleben, Germany, when grown in the field. All observations were recorded during the plant's dormant season (April) unless otherwise noted. The color determination is in accordance with the 1995 R.H.S. Colour Chart of The Royal Horticultural Society, London, England, except where general color terms of ordinary dictionary significance are used.

Botanical classification: ‘MBS 7002’ is a fertile hybrid of a cross from Miscanthus sinensis and Miscanthus sacchariflorus
Common name: ‘MBS 7002’ Miscanthus
Parentage: polycross of M. sacchariflorus and several M. sinensis

### General Description:

Blooming period: ‘MBS 7002’ may bloom in late fall in the southern and central US. Blooms are retained over the winter.
Plant habit: herbaceous, tuft forming, biomass grass with upright culms. 15-17 leaves per culm.
Height and spread: Top leaf height about 2.7 meters.
Hardiness: Productive growth in Klein-Wanzleben (north central), Germany and Ontario, Canada.
Culture: best in sandy loam, well-drained soil, higher yields at higher soil fertility.
Diseases and pests: No susceptibility or resistance to diseases or pests that affect Miscanthus have been observed in field conditions.
Root description: Fibrous, well branched and dense. Fast-developing creeping rhizomes, with shoots arising 5-10 cm from base of the culms.

### Growth and Propagation:

Propagation: By culm division, in vitro culture, from rhizomes, meristem or auxilliary buds (nodes).
Growth rate: Vigorous.
   Culm (stem) description:
General: Cylindrical, pithy, reed-like, erect, sheathed.
Culm aspect: Rigid and held erect, none are cascading.
Culm color (dormant season): yellowish, lower internodes partly reddish. Midsummer color is green yellowish, lower internodes partly reddish.
Culm size: Average about 0.7 cm in diameter, culm circumference: 2.1 cm, and up to about 2.68 m in height
Basal circumference 193 cm
Compressed circumference: 43.2 cm
Culm surface: Culm is covered with many hairs on the leaf sheaths
Internode length: 6 to 20 cm
Ligule: Membranous, about 4 mm (M×g is 2.5-3 mm), color reddish, 145C, border 59D, longest hair is 2 mm (M×g 1 mm), encircles the entire culm, inner surface is glabrous, hairs on the outer surface, long hairs are mainly on the side, hairs on the side are approximately 8 mm (gig 4-5 mm)
   Foliage description:
Leaf shape: Linear
Leaf base: sheathed
Leaf division: Simple
Leaf Apex: Acuminate
Leaf aspect: Emerging leaves are erect, blades are convex, leaf angle younger leaves 50°, leaf angle older leaves 5°, color code NN155B
Leaf tip younger leaves: ½ pendent
Leaf venation: Parallel, upper surface concave, lower surface convex
Leaf margins: Entire, visible, sharp short bristles under the microscope
Leaf size: Up to 100 cm, width: 2-3.2 cm
Leaf attachment: Sheathed
Leaf arrangement: Alternate, tapering
Leaf surface: Upper-light glossy, lower-matte, single hairs on some leaves on the lower surface
Leaf color (during growing season): Green, no stripes, range between 146A-147A
   Flower description:
General description.—Compact, fan-shaped panicle terminating from each culm in mid to late September, composed of numerous slender, silky aggregate racemes
Lastingness of inflorescence.—Panicles are persistent from fall through winter.
Fragrance.—None.
Panicle size.—Average of 22 cm in length and 31 cm in width.
Angle of raceme: 30°
Panicle color.—Varies from 152D-176B
Spikelet description.—Spikelet in pairs.
Spikelet size.—About 5 mm in length and 1 mm in width (excluding hairs).
Spikelet color: 152C
Spikelet hairs.—12 mm in length, 158C in color.
Awn size: 1 mm
   Reproductive organ description:
Androecium—Anthers; 3, 5 mm in length and 0.5 mm in width, red in color, 187B
Gynoecium—stigma color is 187A, red, 4 mm in length and 0.5 mm in width,
Caryopsis—produces fertile seeds.

‘MBS 7003’ (aka ‘Columbia’).

The following traits have been repeatedly observed and are determined the basic characteristics of ‘MBS 7003,’ which in combination distinguish this Miscanthus hybrid from the known Miscanthus×giganteus and other ornamental M. sinensis forms.

1. Vigorous growth

2. Top leaf height about 2.6 meters

3. Green leaves, no colored stripes are present

4. High biomass yield

5. High tiller density

‘MBS 7003’ can be distinguished from the Miscanthus cultivars ‘Strictus,’ ‘Super Stripe,’ ‘Gold Bar,’ ‘Little Zebra’ and ‘Mysterious Maiden’ in that ‘MBS 7003’ has no stripes or colored bands on its leaves.

In side by side comparisons conducted in Klein-Wanzleben, Germany, ‘MBS 7003’ is more vigorous than either of its parent plants and produces more biomass than either parent. It is late ripening and shows excellent winter survival. The leaves stay longer on the culm compared to M.×giganteus and, therefore, the leaf loss during the winter is minimized which, in turn, leads to higher biomass yield. ‘MBS 7003’ develops inflorescences and viable seeds under optimal growing conditions.

The plant can be propagated by rhizomes, from meristem or nodes. This further distinguishes ‘MBS 7003’ from M. sinensis in that M. sinensis cannot be propagated by nodes.

‘MBS 7003’ has not been observed under all possible environmental conditions, and the phenotype may vary significantly with variations in environment. The following observations, measurements, and comparison describe this plant as grown at Klein-Wanzleben, Germany, when grown in the field. All observations were recorded during the plant's dormant season (April) unless otherwise noted. The color determination is in accordance with the 1995 R.H.S. Colour Chart of The Royal Horticultural Society, London, England, except where general color terms of ordinary dictionary significance are used.

Botanical classification: ‘MBS 7003’ is a fertile hybrid of a cross from Miscanthus sinensis and Miscanthus sacchariflorus
Common name: ‘MBS 7003’ Miscanthus
Parentage: polycross of M. sacchariflorus and several M. sinensis

### General Description:

Plant habit: herbaceous, tuft forming, biomass grass with upright culms. 16-21 leaves on the culm.
Height and spread: Top leaf height about 2.6 meters.
Hardiness: Productive growth in Klein-Wanzleben (north central), Germany
Culture: best in sandy loam, well-drained soil, higher yields in warmer climates and higher soil fertility.
Diseases and pests: No susceptibility or resistance to diseases or pests that affect Miscanthus has been observed in field conditions.

### Growth and Propagation:

Propagation: by culm division, in vitro culture, from rhizomes, meristem or auxilliary buds (nodes).
Growth rate: Vigorous.
Culm (stem) description:
General: Cylindrical, pithy, reed-like, erect, sheathed.
Culm aspect: Rigid and held erect, none are cascading.
Culm color (dormant season): yellowish, lower internodes partly reddish, Midsummer color is green yellowish, lower internodes partly reddish
Culm size: Average about 0.59 cm in diameter
Culm surface: Culm is covered with hairs in proximity to the leaf sheaths
Culm circumference: 2.5 cm
Basal circumference: 137.2 cm
Plant compressed circumference: 25.4 cm
Internode length: 6 to 18 cm
Ligule: Membranous, about 3 mm (M×g is 2.5-3 mm), reddish color 59B, longest hair is 2.5 mm (M×g 1 mm), encircles the entire culm, inner surface is glabrous, hairs on the outer surface, long hairs are mainly on the side, hairs on the side are approximately 8 mm (M×g 4-5 mm)
   Foliage description:
Leaf shape: Linear
Leaf base: sheathed
Leaf division: Simple
Leaf Apex: acuminate,
Leaf aspect: Emerging leaves are erect, blades are convex, leaf angle younger leaves 50°, leaf angle older leaves 5°.
Leaf tip younger leaves: ⅔ pendent
Leaf venation: Parallel, main venation concave upper leaf surface, convex lower leaf surface, mid-rib color is whitish on upper surface, color: 155B. Venation aspect: ripply.
Leaf margins: Entirely visible, sharp short bristles under the microscope
Leaf size: Up to 90 cm, width: 2-2.8 cm
Leaf attachment: Sheathed
Leaf arrangement: Alternate, tapering
Leaf surface: Upper-light glossy, lower-matt
Leaf color (during growing season): Green, no stripes, 137B
No hairs on lower and upper leaf surface, a few hairs only near the ligula on upper surface
   Flower description:
General description.—Flowers observed at greenhouse in Klein-Wanzleben, Germany. symmetric arrangement, Compact, fan-shaped panicle terminating from each culm in mid to late September, composed of numerous slender, silky aggregate racemes.
Lastingness of inflorescence.—Panicles are persistent from fall through winter, in greenhouse at Klein-Wanzleben, Germany
Fragrance.—None.
Panicle size.—Average of 41 cm in length and 44 cm in width.
Panicle color.—green, 151A
Angle of raceme: 45°
Spikelet description.—awn 1 mm beyond spikelet
Spikelet color: 163B
Spikelet size.—About 4 mm in length and 1 mm in width (excluding hairs).
Spikelet hairs.—average of 10 mm in length, 158C in color.
   Reproductive organ description:
Androecium.—Anthers; 2 mm in length and 0.5 mm in width, reddish, 187A in color,
Gynoecium.—stigma color is 187B, red, 3 mm in length and 0.5 mm in width,
Caryopsis.—fertile seeds develop.

‘MBS 1001’ (aka ‘MBS 7004’).

The following traits have been repeatedly observed and are determined the basic characteristics of ‘MBS 1001,’ which in combination distinguish this Miscanthus hybrid from the known Miscanthus×giganteus and other ornamental M. sinensis forms.

1. Vigorous growth

2. Top leaf height of about 2.6 meters

3. Green leaves, no colored stripes are present

4. High biomass yield

5. High tiller density

‘MBS 1001’ can be distinguished from the Miscanthus cultivars ‘Strictus,’ ‘Super Stripe,’ ‘Gold Bar,’ ‘Little Zebra’ and ‘Mysterious Maiden’ in that ‘MBS 1001’ has no stripes or colored bands on its leaves.

In side by side comparisons conducted in Klein-Wanzleben, Germany, ‘MBS 1001’ is more vigorous than either of its parent plants and produces more biomass than either parent. Some leaves stay longer on the top of the culm compared to M.×giganteus during winter. ‘MBS 1001’ develops inflorescences and viable seeds under optimal growing conditions.

The plant can be propagated by rhizomes, from meristem or nodes. This further distinguishes ‘MBS 1001’ from M. sinensis in that M. sinensis cannot be propagated by nodes.

‘MBS 1001’ has not been observed under all possible environmental conditions, and the phenotype may vary significantly with variations in environment. The following observations, measurements, and comparison describe this plant as grown at Klein-Wanzleben, Germany, when grown in the field. All observations were recorded during the plant's dormant season (April) unless otherwise noted.

Botanical classification: ‘MBS 1001’ is a fertile hybrid of a cross from Miscanthus sinensis and Miscanthus sacchariflorus
Common name: ‘MBS 1001’ Miscanthus
Parentage: polycross of M. sacchariflorus and several M. sinensis

### General Description:

Blooming period: Flowering was not observed at Klein-Wanzleben (north central), Germany
Plant habit: herbaceous, tuft forming, biomass grass with upright culms
Height and spread: Top leaf height about 2.0 meters.
Hardiness: Productive growth in Klein-Wanzleben (north central), Germany.
Culture: best in sandy loam, well-drained soil, higher yields in warmer climates and higher soil fertility.
Diseases and pests: no susceptibility or resistance to diseases or pests that affect Miscanthus has been observed.

### Growth and Propagation:

Propagation: By culm division, in vitro culture, from rhizomes, meristem or auxilliary buds (nodes).
Growth rate: Vigorous.
   Culm (stem) description:
General: Cylindrical, pithy, reed-like, erect, sheathed. 15-17 leaves per culm.
Culm aspect: Rigid and held erect, none are cascading.
Culm color (dormant season): Yellowish, lower internodes partly reddish. Midsummer color is green yellowish, lower internodes partly reddish
Culm size: Average about 0.51 cm in diameter, up to about 2.4 cm in circumference and up to about 2.6 m in height on mature plants
Plant Basal circumference: 137.2 cm
Plant compressed circumference: 20.3 cm
Culm surface: Culm is covered with hairs on the leaf sheaths covering the culm
Internode length: 6 to 18 cm
Ligule: Membranous, about 2.5 mm (M×g is 2.5-3 mm), reddish color 145C, border 59D, longest hair is 2 mm (M×g 1 mm), encircles the entire culm, inner surface is glabrous, single hairs on the outer surface, long hairs are over the entire ligule, hairs are approximately 2 mm (M×g 4-5 mm)
   Foliage description:
Leaf shape: Linear
Leaf base: sheathed
Leaf division: Simple
Leaf Apex: acuminate
Leaf aspect: Emerging leaves are erect, blades are convex, leaf angle younger leaves 50°, leaf angle older leaves 10°.
Leaf venation: Parallel, upper surface concave, lower surface concex, upper surface venation whitely, NN155B. Venation aspect: ripply
Leaf margins: Entirely visible, sharp short bristles under the microscope
Leaf size: Up to 85 cm, width: 2-2.5 cm
Leaf persistence: foliage dries and is generally retained on the stem during winter
Leaf attachment: Sheathed
Leaf arrangement: Alternate, tapering
Leaf surface: Upper-light glossy, lower-matte. No hairs on upper and lower leaf surface, on upper surface hairs near the ligula only.
Leaf color (during growing season): Green, no stripes, 137B
   Flower description:
General description.—arrangement symmetric, spikelets parallel
Lastingness of inflorescence.—Panicles tend to be persistent from fall through winter
Fragrance.—None.
Angle of raceme: 45°
Panicle size.—Average of 36 cm in length and 35 cm in width.
Panicle color.—green 151A (at the time of evaluation)
Spikelet description.—in pairs and parallel
Spikelet color: 199A
Spikelet size.—About 4 mm in length and 1 mm in width (excluding hairs).
Awn: 1 mm
Spikelet hairs.—12 mm in length, 158C in color.
   Reproductive organ description:
Androecium.—Anthers; 2 mm in length and 0.5 mm in width, reddish 187A in color.
Gynoecium.—stigma color is 187B, 3 mm in length and 0.5 mm in width.
Caryopsis.—produces fertile seeds.

‘MBS 1002’ (aka ‘MBS 7005’).

The following traits have been repeatedly observed and are determined the basic characteristics of ‘MBS 1002,’ which in combination distinguish this Miscanthus hybrid from the known Miscanthus×giganteus and other ornamental M. sinensis forms.

1. Vigorous growth
2. Top leaf height about 2.6 meters
3. Green leaves, no colored stripes are present
4. High biomass yield
5. High tiller density

‘MBS 1002’ can be distinguished from the Miscanthus cultivars Strictus, Super Stripe, Gold Bar, Little Zebra and Mysterious Maiden in that ‘MBS 1002’ has no stripes or colored bands on its leaves.

In side by side comparisons conducted in Klein-Wanzleben, Germany, ‘MBS 1002’ is more vigorous than either of its parent plants and produces more biomass than either parent. ‘MBS 1002’ has taller culms but demonstrates less lodging; hence it has stronger culms.

The plant can be propagated by rhizomes, from meristem or nodes. This further distinguishes ‘MBS 1002’ from M. sinensis in that M. sinensis cannot be propagated by nodes.

‘MBS 1002’ has not been observed under all possible environmental conditions, and the phenotype may vary significantly with variations in environment. The following observations, measurements, and comparison describe this plant as grown at Klein-Wanzleben, Germany, when grown in the field. All observations were recorded during the plant's dormant season (April) unless otherwise noted.

Botanical classification: ‘MBS 1002’ is a fertile hybrid of a cross from Miscanthus sinensis and Miscanthus sacchariflorus.
Common name: ‘MBS 1002’ Miscanthus
Parentage: polycross of M. sacchariflorus and several M. sinensis

### General Description:

Blooming period: ‘MBS 1002’ blooms in late fall in the southern and central US. Blooms at the end of September in Klein-Wanzleben (north central), Germany. Blooms are retained over the winter.
Plant habit: herbaceous, tuft forming, biomass grass with upright culms.
Height and spread: Top leaf height about 2.6 meters.
Hardiness: Productive growth in Klein-Wanzleben (north central), Germany
Culture: best in sandy loam, well-drained soil, higher yields in warmer climates and higher soil fertility.
Diseases and pests: No susceptibility or resistance to diseases or pests that affect Miscanthus has been observed in field conditions.

### Growth and Propagation:

Propagation: by culm division, in vitro culture, from rhizomes, meristem or auxilliary buds (nodes).
Growth rate: Vigorous.
   Culm (stem) description:
Genera description: Cylindrical, pithy, reed-like, erect, sheathed. 15-17 leaves per culm
Culm aspect: Rigid and held erect, none are cascading.
Culm color (dormant season): yellowish, lower internodes partly reddish. Midsummer color is green yellowish.
Culm size: Average about 0.73 cm in diameter, and up to about 2.6 m in height on mature plants
Culm circumference: 2.8 cm
Plant basal circumference: 193 cm
Plant compressed circumference: 38.1 cm
Culm surface: Culm is covered with a few hairs on the leaf sheaths
Internode length: 6 to 18 cm
Ligule: Membranous, about 3 mm (M×g is 2.5-3 mm), reddish color 59D, longest hair is 1.5 mm (M×g 1 mm), encircles the entire culm, inner surface is glabrous, hairs on the outer surface, on entire ligule, hairs are approximately 4 mm (M×g 4-5 mm)
   Foliage description:
General: No hairs on upper and lower leaf surface, some larger hairs on upper surface near ligula
Leaf shape: Linear
Leaf base: sheathed
Leaf division: Simple
Leaf Apex: acuminate
Leaf aspect: Emerging leaves are erect, blades are convex, leaf angle younger leaves 50°, leaf angle older leaves 10°.
Leaf tip younger leaves: ½ pendent
Leaf venation: Parallel, leaf venation upper surface concave, lower surface convex, mid-rib color is whitish.
Leaf margins: Entire, visible, sharp short bristles under the microscope
Leaf size: Up to 90 cm, width: 2-2.8 cm
Leaf persistence: Foliage dries and is generally retained on the stem during winter
Leaf attachment: Sheathed
Leaf arrangement: Alternate, tapering
Leaf surface: Upper-light glossy, lower-matte
Leaf color (during growing season): Green, no stripes, 146A
   Flower description:
General description.—Compact, fan-shaped panicle terminating from each culm in mid to late September
Angle of raceme: 45°
Lastingness of inflorescence.—Panicles are persistent from fall through winter.
Fragrance.—None.
Panicle size.—Average of 36 cm in length, not completely emerged at time of measurement, 17 cm in width (data from one location).
Panicle color.—153C-174B
Spikelet description.—Spikelets in pairs, awn: 2 mm
Spikelet size.—About 4 mm in length and 1 mm in width (excluding hairs).
Spikelet hairs.—Average of 12 mm in length, 186B in color.
Spikelet color: 181A
   Reproductive organ description:
Androecium.—Anthers; 3 mm in length and 0.5 mm in width, 187A or 4C in color, reddish or yellow
Gynoecium.—Stigma color is 187A, red, 3 mm in length and 0.5 mm in width
Caryopsis.—Produces fertile seeds

This invention further relates to plant parts from a fertile, high biomass yielding Miscanthus plant, for example, a plant of varieties ‘MBS 7002,’ ‘MBS 7003,’ ‘MBS 1001,’ ‘MBS 1002,’ including cells and protoplasts, anthers, pistils, stamens, pollen, ovules, flowers, embryos, stems, buds, cotyledons, hypocotyls, roots including root tips and root hairs, rhizomes leaves, seeds, microspores and vegetative parts, whether mature or embryonic. This invention also relates to the use of these plant parts for regenerating plants. The plant parts (e.g., rhizomes or other plant parts), seeds, cells, tissue culture, etc. may be used to regenerate plants having substantially all the improved morphological and physiological characteristics of the selected Miscanthus varieties described herein.

In some embodiments, the present invention provides tissue culture material or cultured cells derived, in whole or in part, from a Miscanthus plant part. One embodiment of the present invention is the clonal multiplication of the Miscanthus plants of the present invention. Methods for clonally increasing Miscanthus via shoot multiplication in culture are well known in the art. See, for example, International Patent Application No. PCT/US2009/051355, filed on Jul. 22, 2009, and published as WO 2010/011717 on Jan. 28, 2010.

Another embodiment is a Miscanthus plant regenerated from such a tissue culture or cultured cells, having the improved morphological and physiological characteristics of the instant Miscanthus varieties described herein. Tissue culture of Miscanthus has been previously described. See, for example, PCT application PCT/US09/41424, hereby incorporated by reference in its entirety, or Yi et al. (2001) High Tech. Lett. 11: 20-24.

This invention further relates to the use of a fertile, high biomass yielding Miscanthus plant, for example, a plant of Miscanthus varieties ‘MBS 7002,’ ‘MBS 7003,’ ‘MBS 1001,’ ‘MBS 1002’ for breeding Miscanthus plants, through pedigree breeding, crossing, self-pollination, haploidy, single seed descent, modified single seed descent, and backcrossing, or other suitable breeding methods, and to the plants produced. This invention also relates to a method for producing a first generation (F1) hybrid Miscanthus seed by crossing one of the plants described above with an inbred plant of a different variety or species, and harvesting the resultant first generation (F1) hybrid seed. It further relates to the plants produced from the F1 hybrid seed.

The invention also relates to plant products derived from a fertile, high biomass yielding Miscanthus plant, for example, a plant of Miscanthus varieties ‘MBS 7002,’ ‘MBS 7003,’ ‘MBS 1001,’ or ‘MBS 1002’ used for fuel or energy capture, energy storage, or energy production. These three patent applications are hereby incorporated by reference in their entirety.

Another aspect of the present invention provides a method for producing Miscanthus seed comprising crossing a first parent Miscanthus plant with a 4× ploidy and a second parent Miscanthus plant of 2× ploidy and harvesting resultant first-generation (F1) hybrid Miscanthus seed, wherein said hybrid Miscanthus seed is one of a fertile, high biomass yielding Miscanthus plant, for example, a plant of Miscanthus varieties ‘MBS 7002,’ ‘MBS 7003,’ ‘MBS 1001,’ or ‘MBS 1002.’

Another aspect of the present invention provides a method for producing Miscanthus seed comprising crossing a first parent Miscanthus plant and a second parent Miscanthus plant and harvesting resultant first-generation (F1) hybrid Miscanthus seed, wherein said first or second parent Miscanthus plant is one of a fertile, high biomass yielding Miscanthus variety such as, but not limited to, ‘MBS 7002,’ ‘MBS 7003,’ ‘MBS 1001,’ or ‘MBS 1002.’

The invention also relates to plants or products produced by manipulating the genome of one of a fertile, high biomass yielding Miscanthus variety such as, but not limited to, ‘MBS 7002,’ ‘MBS 7003,’ ‘MBS 1001,’ or ‘MBS 1002,’ such as, for example, by genetically transforming or mutagenizing plants or plant parts of Miscanthus varieties ‘MBS 7002,’ ‘MBS 7003,’ ‘MBS 1001,’ or ‘MBS 1002.’

Manipulating the genome of one of the instant fertile, high biomass yielding Miscanthus varieties can be performed to produce various phenotypes of agronomic interest, such as greater disease resistance, insect resistance, herbicide resistance, improved biomass, improved water deficit tolerance, altered lignin content, and the like. Transformation can also be used to insert DNA sequences which control or help control male-sterility. DNA sequences native to Miscanthus as well as non-native DNA sequences can be transformed into Miscanthus and used to alter levels of native or non-native proteins. Various promoters, targeting sequences, enhancing sequences, and other DNA sequences can be inserted into the Miscanthus genome for the purpose of altering the expression of proteins. Reduction of the activity of specific genes (also known as gene silencing, or gene suppression) is desirable for several aspects of genetic engineering in plants. See, for example, U.S. Patent Application Publication No. US20060282918 and its cited references; WO 2009/132116, published Oct. 29, 2009; WO 2010/065534, published Jun. 10, 2010, for further details of methods described in this and above paragraphs, for example, transformation procedures, promoters, reduction of gene activity, etc.

In some embodiments, the present invention also provides Miscanthus varieties, hybrids and synthetic populations that can be utilized for genomic testing according to methods well known to those skilled in the art. See, for example, Swaminathan et al. (2010) Genome Biology 11:R12, 1-18 and Atienza et al. (2003) Theor Appl Genet 107(1):123-130.

In some embodiments, this invention provides fertile, tetraploid varieties of Miscanthus, wherein the fertile, tetraploid varieties of Miscanthus have greater seedling vigor than Miscanthus sinensis, greater vigor than the M. sinensis or Miscanthus×giganteus Greef et Deu ex. Hodkinson et Renvoize (“M×g”) ‘Illinois’ clone, greater tolerance to water deficit than the M×g ‘Illinois’ clone, greater tolerance to cold than the M. sinensis, or is capable of producing a percentage of a yield of biomass produced by the M×g ‘Illinois’ clone, when the tetraploid variety and the M×g ‘Illinois’ clone are at substantially the same stage of seedling or plant development having been grown under substantially the same environmental conditions, wherein the percentage is selected from the group consisting of at least 75%, at least 80%, at least 85%, at least 90%, at least 95%, at least 100%, at least 105%, at least 110%, at least 115%, at least 120%, at least 125% or more.

In some embodiments, this invention provides such fertile, tetraploid varieties of Miscanthus, wherein the yield of biomass of the M×g ‘Illinois’ clone or the fertile, tetraploid varieties of Miscanthus are at least 10, at least 15, at least 20, at least 25, at least 27, at least 30, at least 33, at least 35, at least 40, at least 44 tonnes or more of dry matter per hectare per year.

In some embodiments, the present invention provides seed obtained from flowers of the fertile, tetraploid varieties of Miscanthus of the present invention, wherein the seed is capable of germinating into a plants that have greater seedling vigor than the Miscanthus sinensis, greater vigor than the Miscanthus sinensis or the M×g ‘Illinois’ clone, greater tolerance to water deficit than the M×g ‘Illinois’ clone, greater tolerance to cold than Miscanthus sinensis, or is capable of producing a greater yield of biomass than the yield of biomass produced by the M×g ‘Illinois’ clone, when the fertile tetraploid varieties and the M×g ‘Illinois’ clone are at substantially the same stage of seedling or plant development having been grown under substantially the same environmental conditions.

In some embodiments, the present invention provides seed obtained from flowers of a second Miscanthus variety, or any other cross-compatible genus, produced as a result of pollination with pollen of a first fertile, tetraploid variety of Miscanthus of the present invention.

In some embodiments, the present invention provides plant cells of the fertile, tetraploid varieties of Miscanthus of the present invention.

In other embodiments, the present invention provides tissue cultures of regenerable cells of the fertile, tetraploid varieties of Miscanthus of the present invention.

In other embodiments, the present invention provides plant parts of the fertile, tetraploid varieties of Miscanthus of the present invention, wherein such plant parts include but are not limited to the biomass of the plants.

In some embodiments, the present invention provides fertile, tetraploid varieties of Miscanthus of the present invention, wherein seedlings of the fertile, tetraploid varieties of Miscanthus are more tolerant to water deficit conditions than seedlings of the M×g ‘Illinois’ clone when both the varieties and the M×g ‘Illinois’ clone are at substantially the same stage of seedling development having been grown under substantially the same environmental conditions.

In some embodiments, the present invention provides the fertile, tetraploid varieties ‘MBS 7002,’ ‘MBS 7003,’ ‘MBS 1001,’ or ‘MBS 1002.’

In other embodiments, the present invention provides seed harvested from a flower of a Miscanthus line designated ‘MBS 7002,’ ‘MBS 7003,’ ‘MBS 1001,’ or ‘MBS 1002.’ In other embodiments, the present invention provides Miscanthus progeny plants produced by such seed and parts of said Miscanthus progeny plants.

In some embodiments, the present invention provides fertile, tetraploid varieties of Miscanthus of the present invention, wherein the fertile, tetraploid varieties of Miscanthus have been selected for the greater seedling vigor than Miscanthus sinensis, greater vigor than Miscanthus sinensis or the M×g ‘Illinois’ clone, greater tolerance to water deficit than the M×g ‘Illinois’ clone, greater tolerance to cold than Miscanthus sinensis, or a greater percentage of biomass yield than that produced by the M×g ‘Illinois’ clone, when the fertile, tetraploid varieties and the M×g ‘Illinois’ clone are harvested at substantially the same stage of development having been grown under substantially the same environmental conditions.

In some embodiments, the present invention provides methods of producing a fertile, tetraploid varieties of Miscanthus, wherein the fertile, tetraploid varieties of Miscanthus have greater seedling vigor than M. sinensis, greater vigor than the Miscanthus sinensis or Miscanthus×giganteus Greef et Deu ex. Hodkinson et Renvoize (“M×g”) ‘Illinois’ clone, greater tolerance to water deficit than the M×g ‘Illinois’ clone, greater tolerance to cold than the Miscanthus sinensis, or are capable of producing a percentage of a yield of biomass produced by the M×g ‘Illinois’ clone, when the fertile, tetraploid varieties and the M×g ‘Illinois’ clone are harvested at substantially the same stage of development having been grown under substantially the same environmental conditions; and the percentage is selected from the group consisting of: at least 75%, at least 80%, at least 85%, at least 90%, at least 95%, at least 100%, at least 105%, at least 110%, at least 115%, at least 120%, at least 125% or more, the method steps including:
a) crossing a tetraploid M. sacchariflorus with a diploid M. sinensis, or a tetraploid M. sinensis with a diploid M. sacchariflorus;
b) identifying a progeny plant that is tetraploid;
c) identifying from amongst a plurality of the progeny plant those that retain fertility when grown adjacent to a different Miscanthus line with a different self-incompatibility group.

In some embodiments, the present invention provides such methods wherein the yield of biomass produced by the M×g ‘Illinois’ clone or the fertile, tetraploid varieties of Miscanthus are at least 10, at least 15, at least 20, at least 25, at least 27, at least 30, at least 33, at least 35, at least 40, at least 44 tonnes or more of dry matter per hectare per year.

In some embodiments, the present invention provides methods of introducing a heritable trait into a Miscanthus plant, wherein the heritable trait is greater seedling vigor than Miscanthus sinensis, greater vigor than the M. sinensis or Miscanthus×giganteus Greef et Deu ex. Hodkinson et Renvoize (“M×g”) ‘Illinois’ clone, greater tolerance to water deficit than the M×g ‘Illinois’ clone, greater tolerance to cold than the Miscanthus sinensis, or production of a percentage of the yield of biomass produced by the M×g ‘Illinois’ clone; wherein the yield of biomass is a percentage of the biomass produced by the M×g ‘Illinois’ clone, and the percentage is selected from the group consisting of: at least 75%, at least 80%, at least 85%, at least 90%, at least 95%, at least 100%, at least 105%, at least 110%, at least 115%, at least 120%, at least 125% or more; the method steps including;

(a) crossing a first Miscanthus plant with a second Miscanthus plant that heritably carries the heritable trait to produce F₁ progeny plants, at least some of which heritably carry the trait;

(b) selecting F₁ progeny plants that heritably carry the trait;

(c) crossing the selected progeny plants with another plant to produce next-generation progeny plants at least some of which heritably carry the trait;

(d) selecting next-generation progeny plants that heritably carry the heritable trait; and optionally

(e) repeating steps (c) and (d) to produce selected progeny plants that comprise the heritable trait.

In some embodiments, the present invention provides use of a seed of Miscanthus varieties to produce Miscanthus plants having greater seedling vigor than M. sinensis, greater vigor than the Miscanthus sinensis or Miscanthus×giganteus Greef et Deu ex. Hodkinson et Renvoize (“M×g”) ‘Illinois’ clone, greater tolerance to water deficit than the M×g ‘Illinois’ clone, greater tolerance to cold than the Miscanthus sinensis, or a percentage of yield of biomass of the biomass produced by the M×g ‘Illinois’ clone; wherein the percentage is selected from the group consisting of: at least 75%, at least 80%, at least 85%, at least 90%, at least 95%, at least 100%, at least 105%, at least 110%, at least 115%, at least 120%, at least 125% or more; said seed produced by crossing a first Miscanthus plant having the greater seedling vigor, the greater vigor, the greater water deficit tolerance, or the greater cold tolerance, with a second Miscanthus plant producing the percentage of the yield of the biomass of the M×g ‘Illinois’ clone.

In some embodiments, the present invention provides fertile, tetraploid Miscanthus plants producing a percentage of the yield of biomass produced by Miscanthus×giganteus Greef et Deu ex. Hodkinson et Renvoize (“M×g”) ‘Illinois’ clone; and the percentage is selected from the group consisting of: at least 75%, at least 80%, at least 85%, at least 90%, at least 95%, at least 100%, at least 105%, at least 110%, at least 115%, at least 120%, at least 125% or more; wherein at least one ancestor of said fertile, tetraploid Miscanthus plants is the Miscanthus plants of the present invention. In other embodiments, the present invention provides the fertile, tetraploid Miscanthus plants of the present invention, wherein the yield of biomass produced by the M×g ‘Illinois’ clone or the fertile, tetraploid Miscanthus plants are at least 10, at least 15, at least 20, at least 25, at least 27, at least 30, at least 33, at least 35, at least 40, at least 44 tonnes or more of dry matter per hectare per year.

In some embodiments, the present invention provides populations of fertile, tetraploid Miscanthus plants, wherein the population is composed of two or more genetically distinct plants; and the two or more genetically distinct plants each are more tolerant to water deficit than Miscanthus×giganteus Greef et Deu ex. Hodkinson et Renvoize (“M×g”) ‘Illinois’ clone, or have greater seedling vigor than Miscanthus sinensis, or have greater vigor than the Miscanthus sinensis or the M×g ‘Illinois’ clone, or are more tolerant to cold than the M. sinensis, or produce a percentage of the yield of biomass produced by a plant of the M×g ‘Illinois’ clone; and the percentage is selected from the group consisting of: at least 75%, at least 80%, at least 85%, at least 90%, at least 95%, at least 100%, at least 105%, at least 110%, at least 115%, at least 120%, at least 125% or more. In some embodiments, the present invention provides such populations of fertile, tetraploid Miscanthus plants wherein the two or more genetically distinct plants are selected from the group consisting of ‘MBS 7002,’ ‘MBS 7003,’ ‘MBS 1001,’ and ‘MBS 1002.’ In some embodiments, the present invention provides such populations of fertile, tetraploid Miscanthus plants wherein the yield of biomass produced by the plant of the M×g ‘Illinois’ clone or the population of fertile, tetraploid Miscanthus plants is at least 10, at least 15, at least 20, at least 25, at least 27, at least 30, at least 33, at least 35, at least 40, at least 44 tonnes or more of dry matter per hectare per year. In some embodiments, the present invention provides seeds harvested from a flower of the population of such fertile tetraploid Miscanthus plants. In some embodiments, the present invention provides Miscanthus progeny plants produced from the seed of such populations of Miscanthus progeny plants, and parts of said Miscanthus progeny plants.

In some embodiments, the present invention provides methods for producing high-biomass Miscanthus progeny plants having greater seedling vigor than Miscanthus sinensis, greater vigor than the Miscanthus sinensis or Miscanthus×giganteus Greef et Deu ex. Hodkinson et Renvoize (“M×g”) ‘Illinois’ clone, greater tolerance to water deficit than the M×g ‘Illinois’ clone, or greater tolerance to cold than the Miscanthus sinensis, or a percentage of yield of biomass of the biomass produced by the M×g ‘Illinois’ clone, the method steps including crossing a first fertile tetraploid high-biomass Miscanthus plant with a second fertile tetraploid high-biomass Miscanthus plant; harvesting seed that results from the crossing; and growing the seed to produce the high-biomass Miscanthus progeny plant; wherein high-biomass is characterized by the percentage of yield of biomass of the biomass produced by the M×g ‘Illinois’ clone, when the progeny Miscanthus plants, the first fertile tetraploid high-biomass Miscanthus plants, the second fertile tetraploid high-biomass Miscanthus plants, or the M×g ‘Illinois’ clone are harvested at substantially the same stage of development having been grown under substantially the same environmental conditions; and the percentage is selected from the group consisting of: at least 75%, at least 80%, at least 85%, at least 90%, at least 95%, at least 100%, at least 105%, at least 110%, at least 115%, at least 120%, at least 125% or more. In some embodiments, the present invention provides such methods wherein the yield of biomass of the progeny Miscanthus plants, the first fertile tetraploid high-biomass Miscanthus plants, the second fertile tetraploid high-biomass Miscanthus plants, or the M×g ‘Illinois’ clone is at least 10, at least 15, at least 20, at least 25, at least 27, at least 30, at least 33, at least 35, at least 40, at least 44 tonnes or more of dry matter per hectare per year. In some embodiments, the present invention provides seeds obtained from the crossing of the first fertile tetraploid high-biomass Miscanthus plant with the second fertile tetraploid high-biomass Miscanthus plant of such methods. In some embodiments the present invention provides seeds obtained from a flower of third Miscanthus plants, or any other cross-compatible genus, produced as a result of pollination with pollen of the high-biomass Miscanthus progeny plants of the present invention. In some embodiments, the present invention provides plant cells, plant parts, or tissue cultures of regenerable cells of the high-biomass Miscanthus progeny plant of the present invention. In some embodiments, the present invention provides biomass comprising the plant parts of the Miscanthus progeny plants of the present invention.

In some embodiments, the present invention provides methods of using the Miscanthus varieties, hybrids, synthetics and open pollinated populations of the present invention for biofuel production. Methods of using plant material (e.g., corn seed, sugarcane or sorghum bagasse) as feedstocks for biofuel production are well known to those skilled in the art. For some specific articles focused on using Miscanthus for biofuel production, see, for example, Vrije et al. (2009) Biotechnology for biofuels 2:12, 1-15; Ligero et al. (2010) Bioresour Technol 101(9):3188-3193; Hage et al. (2010) Bioresour Technol 101(23):9321-9329; and Villayerde et al. (2009) J Agric Food Chem 57(9):3626-3631.

### DEPOSIT INFORMATION

A deposit of seeds of the following four crosses representative of this invention is maintained by Mendel BioEnergy Seeds, a division of Mendel Biotechnology, Inc.: (1) ‘MBS 7002’×‘MBS 7004;’ (2) ‘MBS 7002’×‘MBS 7005;’ (3) ‘MBS 7004’×‘MBS 7005;’ and (4) ‘MBS 7002’×‘MBS 7004’×‘MBS 7005.’ In addition, a sample of seed of each of these four crosses is presently being deposited with the American Type Culture Collection (ATCC), 10801 University Boulevard, Manassas, Va. 20109.

To satisfy the enablement requirements of 35 U.S.C. 112, and to certify that the deposit of the seeds of the present invention meets the criteria set forth in 37 CFR 1.801-1.809, Applicants hereby make the following statements regarding the deposited seeds:

1. During the pendency of this application, access to the invention will be afforded to the Commissioner upon request;

2. Upon granting of the patent the strain will be available to the public under conditions specified in 37 CFR 1.808;

3. The deposit will be maintained in a public repository for a period of 30 years or 5 years after the last request or for the enforceable life of the patent, whichever is longer; and

4. The viability of the biological material at the time of deposit will be tested; and

5. The deposit will be replaced if it should ever become unavailable.

Access to this deposit will be available during the pendency of this application to persons determined by the Commissioner of Patents and Trademarks to be entitled thereto under 37 C.F.R. §1.14 and 35 U.S.C. §122. Upon allowance of any claims in this application, all restrictions on the availability to the public of the variety will be irrevocably removed by affording access to a deposit of at least 2,500 seeds of the same variety with the ATCC.

### EXAMPLES

### Example I

### Generation of Novel Miscanthus Lines Via Interspecific Crosses

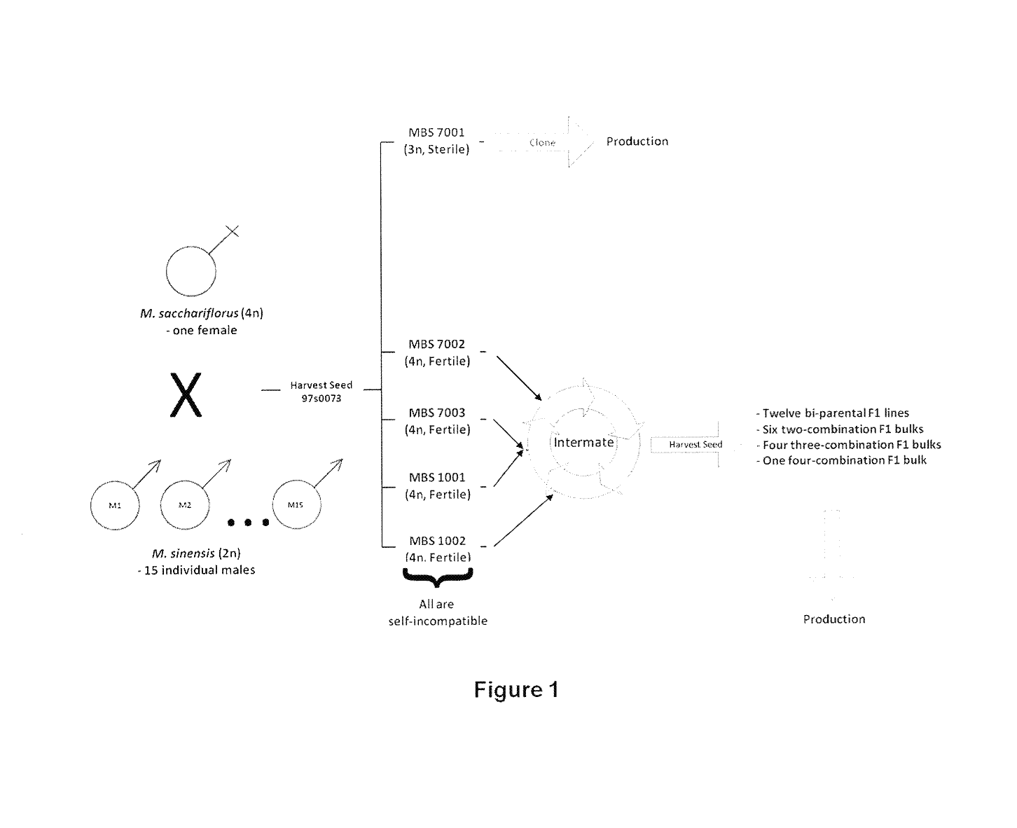

Miscanthus varieties were generated by crossing a large-stemmed M. sacchariflorus genotype from Japan (ploidy: 4×) as a female parent with a population of 15 M. sinensis (ploidy: 2×) plants as pollen donors. From this cross (designated 97s0073), 158 seedlings were obtained and planted in a field. Based on field observations, five selections for high-biomass were made, one of which was triploid, and four were FTMG varieties. The left-hand side of FIG. 1 provides a schematic of the breeding process used to create ‘MBS 7001,’ ‘MBS 7002,’ ‘MBS 7003,’ ‘MBS 1001’ and MBS 1002.

FTMG varieties could also be produced via induced tetraploidy in diploid parents or progenies. Induced tetraploid genotypes can be obtained by doubling the chromosome number of diploid genotypes using published methods (Glowacka et al. (2009). Industrial Crops Products, 30: 444-446; Petersen et al. (2003) Plant Cell Tissue Organ Culture 73: 137-146; Petersen et al. (2002) Plant Breeding 121: 445-450). For example, a tetraploid M. sacchariflorus genotype from Japan could be crossed with an induced-tetraploid M. sinensis to obtain FTMG varieties. Though M. sacchariflorus genotypes in Japan are primarily triploid, on mainland Asia this species is predominantly diploid, like M. sinensis. We have also obtained fertile diploid progeny from crosses between diploid M. sacchariflorus and diploid M. sinensis. The chromosome number of diploid progeny derived from diploid M. sacchariflorus and diploid M. sinensis could be doubled to obtain FTMG varieties. Alternatively, the chromosome numbers of the diploid M. sacchariflorus and M. sinensis parents could be doubled prior to crossing in order to obtain FTMG varieties.

FTMG varieties produced by these methods, including ‘MBS 7002,’ ‘MBS 7003,’ ‘MBS 1001,’ or ‘MBS 1002,’ are described in U.S. Provisional Patent Application No. 61/050,162, filed May 2, 2008; U.S. patent application Ser. No. 12/387,437 (filed May 1, 2009); U.S. patent application Ser. No. 12/387,429, filed May 1, 2009; and U.S. patent application Ser. No. 12/584,496, filed Sep. 4, 2009. Each and every one of these patent applications are hereby incorporated by reference in their entirety for all purposes.

Control plants used as comparators of biomass yield, water deficit tolerance, seedling vigor or other traits may include Miscanthus×giganteus (M×g), also known as Giant Miscanthus the M×g ‘Illinois’ clone. M×g is well known and readily available to the public. M×g is described in a number of publications, including but not limited to “M×g ‘Illinois’ clone” of the species Miscanthus×giganteus Greef et Deu ex. Hodkinson et Renvoize; Heaton et al. (2008a) Curr. Opin. Biotechnol. 19: 202-209 and Heaton et al. (2008b) Global Change Biol. 14: 2000-2014. Furthermore, M×g is commercially available from a number of sources, including but not limited to New Energy Farms Group, Agrotrader.co.uk. and Victoriana Nursery Gardens.

### Example II

### Morphological and Physiological Attributes of FTMGs

Morphological Characteristics.

FTMG F1 plants were more vigorous and taller than either of their M. sacchariflorus or M. sinensis parents. Tiller density (stems/m²) was greater for FTMG varieties than M. sacchariflorus or the M×g ‘Illinois’ clone. The combination of greater vigor and height than parental lines and higher tiller density than M. sacchariflorus or the M×g ‘Illinois’ clone. FTMG varieties thus conferred to the latter plants relatively high biomass. FTMG varieties also flowered later than the M. sinensis parents, a characteristic that contributed to their greater height.

Improved Seedling Vigor, Cold Tolerance, and Water Deficit Tolerance of FTMG Varieties.

Miscanthus was evaluated in greenhouses and in field trials at different sites spread across two distinct regions in North American. In these trials, FTMG varieties, and particularly those of the instant invention, demonstrated a number of advantages when compared to other fertile species of Miscanthus.

FTMG F2 seedlings were markedly more vigorous than M. sinensis seedlings. This difference in seedling vigor was observed on very young plants growing in cell-trays in a greenhouse and continued after transplanting in the field throughout the first growing season.

Miscanthus×giganteus has been shown to have less water use efficiency than M. sinensis or M. sacchariflorus at the young vegetative stage (Clifton-Brown et al. (2000) Ann. Botany 86: 191-200). However, FTMG varieties ‘MBS 7002,’ ‘MBS 7003,’ ‘MBS 1001,’ or ‘MBS 1002’ grown in Alabama and Mississippi were observed to be more vigorous than M×g. Since water was limiting at various times during the establishment year, one explanation for the greater vigor of the FTMG varieties relative to the M×g ‘Illinois’ clone is that the four FTMG varieties are more tolerant to water deficit tolerance or better at avoiding water deficit. FTMG varieties ‘MBS 7002,’ ‘MBS 7003,’ ‘MBS 1001,’ or ‘MBS 1002’ may thus have greater water use efficiency than the M×g ‘Illinois’ clone. One could thus select for improved yield of FTMG varieties by identifying plants that have greater vigor or that are tolerant to water deprivation and/or have greater water use efficiency than control plants (e.g., plants of the M×g ‘Illinois’ clone).

In comparison with M. sinensis species, enhanced cold tolerance of FTMG varieties is an important advantage. This has been shown with specific varieties of the instant invention. ‘MBS 7002’ and ‘MBS 7003’ were planted in two relatively cold regions in Ontario, Canada, where the lowest air temperature recorded in the first year of growth of these varieties was as low as −24.5° C. to −28.2° C. for the two locations, respectively. At each of these locations ‘MBS 7002’ and ‘MBS 7003’ exhibited a survival rate that was significantly greater than the survival of M. sinensis established at the same time in these locations. These results demonstrate that FTMG varieties are more cold tolerant than another fertile species of Miscanthus, M. sinensis, including during the first year of growth when the plants would be particularly sensitive to cold.

### Example III

### Improved Yield Produced by FTMG Varieties

Miscanthus varieties are expected to develop significantly more biomass than many other plants considered as feedstock candidates, including switchgrass. For example, in an experimental field trial conducted in ‘Illinois,’ Miscanthus×giganteus yielded approximately twice the biomass as switchgrass.

Miscanthus varieties ‘MBS 7002,’ ‘MBS 7003,’ ‘MBS 1001,’ and ‘MBS 1002’ have also consistently exhibited vigorous growth, a top leaf height of about 2.6 meters, and high tiller density relative to many other Miscanthus varieties. In side-by-side comparisons conducted in Klein-Wanzleben, Germany, Miscanthus varieties ‘MBS 7002,’ ‘MBS 7003,’ ‘MBS 1001,’ and ‘MBS 1002’ were more vigorous than either of their parent plants, including with regard to greater seedling vigor than the parent plants, and produced more biomass than either parent. ‘MBS 7002’ and ‘MBS 1002’ had taller culms but demonstrated less lodging; hence they produced stronger culms. Generally, the leaves of these varieties stayed on the culm longer than M.×giganteus controls and, therefore, the leaf loss during the winter is minimized which, in turn, may lead to higher biomass yield. ‘MBS 7003’ was late developing and showed excellent winter survival. These FTMG varieties developed inflorescences and viable seeds under optimal growing conditions.

In a field trail in Kentucky conducted in 2008, FTMG varieties ‘MBS 7002’ and ‘MBS 7003’ each produced yields comparable to the M×g ‘Illinois’ clone grown in the same areas.

Similar superior yields are thus expected to be obtained with Miscanthus varieties ‘MBS 7002,’ ‘MBS 7003,’ ‘MBS 1001,’ ‘MBS 1002,’ which are more water deficit tolerant than Miscanthus×giganteus, and, unlike Miscanthus×giganteus, have the significant benefit of being fertile. Because of these characteristics relative to Miscanthus×giganteus, it is expected that Miscanthus varieties ‘MBS 7002,’ ‘MBS 7003,’ ‘MBS 1001,’ and ‘MBS 1002’ will produce a high biomass yield superior to ornamental M. sinensis varieties and similar or greater to that of Miscanthus×giganteus, on the order of at least 75% to at least 125% or more of the biomass produced by a control Miscanthus×giganteus plant, for example, the M×g ‘Illinois’ clone (Heaton et al., 2008a, 2008b, supra), when varieties ‘MBS 7002,’ ‘MBS 7003,’ ‘MBS 1001,’ or ‘MBS 1002’ and the control plant are harvested at substantially the same stage of development having been grown under substantially the same environmental conditions.

### Example IV

### Propagation of Fertile Miscanthus Varieties

Miscanthus varieties ‘MBS 7002,’ ‘MBS 7003,’ ‘MBS 1001,’ and ‘MBS 1002’ can be propagated from rhizomes, meristems, nodes, or other vegetative tissues in which the genetic composition of the propagated plants are the same as the plants from which the tissues are derived. FTMG seed can be produced from any combination of FTMG parental lines ‘MBS 7002,’ ‘MBS 7003,’ ‘MBS 1001,’ and ‘MBS 1002’ by establishing fields containing these combinations of ‘MBS 7002,’ ‘MBS 7003,’ ‘MBS 1001,’ and ‘MBS 1002’ that have been propagated from rhizomes, meristems, nodes, or other vegetative tissues in which the genetic composition of the propagated plants are the same as the plants from which the tissues are derived.

Selection of Improved FTMG Lines and Yield that may be Obtained from Crossing FTMG Varieties.

In a field trial conducted in Kentucky in 2008, the yield obtained from several cultivars of Miscanthus derived from FTMG seed was compared to the control M×g ‘Illinois’ clone. The FTMG seed was produced from crosses of FTMG parental lines ‘MBS 7002,’ ‘MBS 7003,’ ‘MBS 1001,’ and ‘MBS 1002.’ The M×g ‘Illinois’ clone control produced the highest yield, but this yield value was not significantly different from the top three experimental polycross families grown from the FTMG seed. FTMG progeny that can be produced from crosses of FTMG varieties, or perhaps between FTMG varieties and other Miscanthus lines or any other cross-compatible genus, may then be selected for increased yield or possibly other desirable characteristics such as delayed flowering, seedling vigor or vigor of more mature plants, cold tolerance or water deficit tolerance.

### Example V

### FTMG Varieties and Planting Density

Because of their ability to be propagated by seed, FTMG varieties and their progeny can be planted more cost effectively than plant lines that are propagated asexually, such as with plugs or rhizomes. Thus, FTMG lines ‘MBS 7002,’ ‘MBS 7003,’ ‘MBS 1001,’ and ‘MBS 1002,’ and seeds derived from these lines, may generally be planted at higher plant densities than sterile varieties with less and effort and cost, resulting in higher yields for the former plants in the first few years of growth. Higher planting density may also be used to compensate for plants lost early to various environmental factors, such as winter kill.

### Example VI

### FTMG Varieties and Genetic Diversity

It is well established that genetic diversity improves the ability of crop plants to resist diseases and pests. The present invention provides at least four distinct FTMG varieties, and progeny derived from these varieties, that have the ability to produce significant biomass in the field. Together, a crop produced with these varieties or from crosses of these varieties would not be encumbered by virtually identical individuals that might allow a disease or pest to take hold.

Miscanthus is self-incompatable. Thus, prior to the generation of genetically distinct FTMG varieties described herein, and in the absence of another compatible plant or clone, there has been no efficient way to produce desirable high-biomass progeny Miscanthus plants from seed. Thus, the presently described FTMG varieties may be used in a novel method to produce high-biomass Miscanthus progeny plants from seed. The first step in this method includes crossing a first fertile tetraploid high-biomass yielding Miscanthus plant (e.g., one of the FTMG varieties) with a second fertile tetraploid high-biomass yielding Miscanthus plant (a different FTMG variety). Seed that result from the crossing may then be harvested and grown to produce the high-biomass progeny Miscanthus plant. Optionally, the high-biomass progeny Miscanthus plant may be selected from a plurality of plants produced by this method on the basis of biomass yield and possibly other properties (e.g., seedling vigor, water deficit tolerance). The high biomass value of the plants produced by this method may be evaluated by comparison to a standard, such as a particular percentage of the yield of the biomass produced by the M×g ‘Illinois’ clone when the progeny Miscanthus plant, the first fertile tetraploid high-biomass Miscanthus plant, the second fertile tetraploid high-biomass Miscanthus plant, or the M×g ‘Illinois’ clone are harvested at substantially the same stage of development having been grown under substantially the same environmental conditions. The percentage of the yield can range from, for example, at least 75%, to at least 80%, at least 85%, at least 90%, at least 95%, at least 100%, at least 105%, at least 110%, at least 115%, at least 120%, and to at least 125% or more.

Seed that may be obtained from the crossing of the first fertile tetraploid high-biomass Miscanthus plant with the second fertile tetraploid high-biomass Miscanthus plant, or from a flower of a different Miscanthus variety, or any other cross-compatible genus, produced as a result of pollination with pollen of the high-biomass Miscanthus progeny plant, are also considered part of the present invention.

Plant cells, plant parts, a tissue culture of regenerable cells, and biomass that may be derived from the high-biomass Miscanthus progeny plant or parts thereof are also considered part of the present invention.

### Example VII

### Generation of Novel Hybrid and Synthetic Varieties of Miscanthus

The right-hand side of FIG. 1 provides a schematic of the breeding process used to create two-way, three-way and four-way Miscanthus lines and bulks by crossing ‘MBS 7002,’ ‘MBS 7003,’ ‘MBS 1001’ and ‘MBS 1002’ in various ways and combinations.

There are twelve different bi-parental F1 lines possible which can be created by crossing each of the four varieties in every possible pair-wise combination and maintaining control of which variety is used as the male ( [symbol] ; pollinator) and which is used as the female ( [symbol] ) in the crosses. The twelve combinations are as follows: MBS 7002 [symbol] ×MBS 7003♀; MBS 7002 [symbol] ×MBS 1001 [symbol] ; MBS 7002 [symbol] ×MBS 1002 [symbol] ; MBS 7003 [symbol] ×MBS 7002 [symbol] ; MBS 7003 [symbol] ×MBS 1001 [symbol] ; MBS 7003 [symbol] ×MBS 1002 [symbol] ; MBS 1001 [symbol] ×MBS 7002 [symbol] ; MBS 1001 [symbol] ×MBS 7003 [symbol] ; MBS 1001 [symbol] ×MBS 1002 [symbol] ; MBS 1002 [symbol] ×MBS 7002 [symbol] ; MBS 1002 [symbol] ×MBS 7003 [symbol] ; and MBS 1002 [symbol] ×MBS 1001 [symbol] .

There are six different two-combination, or two way, F1 bulks possible which can be created by crossing each of the four varieties with each other without regard to maintaining control of which variety is used as the male and which is used as the female in the crosses. The six combinations are as follows: ‘MBS 7002’×MBS 7003; ‘MBS 7002’×MBS 1001; ‘MBS 7002’×MBS 1002; ‘MBS 7003’×MBS 1001; ‘MBS 7003’×MBS 1002; and ‘MBS 1001’×MBS 1002.

There are four different three-combination, or 3-way, F1 bulks possible which can be created by intercrossing/intermating any three of the four varieties without regard to maintaining control of which variety is used as the male and which is used as the female in the crosses. The four combinations are as follows: ‘MBS 7002’×‘MBS 7003’×MBS 1001; ‘MBS 7002’×‘MBS 7003’×MBS 1002; ‘MBS 7002’×‘MBS 1001’×MBS 1002; and ‘MBS 7003’×‘MBS 1001’×MBS 1002.

There is one four-combination, or 4-way, F1 bulk possible which can be created by intercrossing/intermating all four of the four varieties without regard to maintaining control of which variety is used as the male and which is used as the female in the crosses. The one combination is as follows: ‘MBS 7002’×‘MBS 7003’×‘MBS 1001’×‘MBS 1002.’

### Example VIII

### Miscanthus Biomass Field Trials

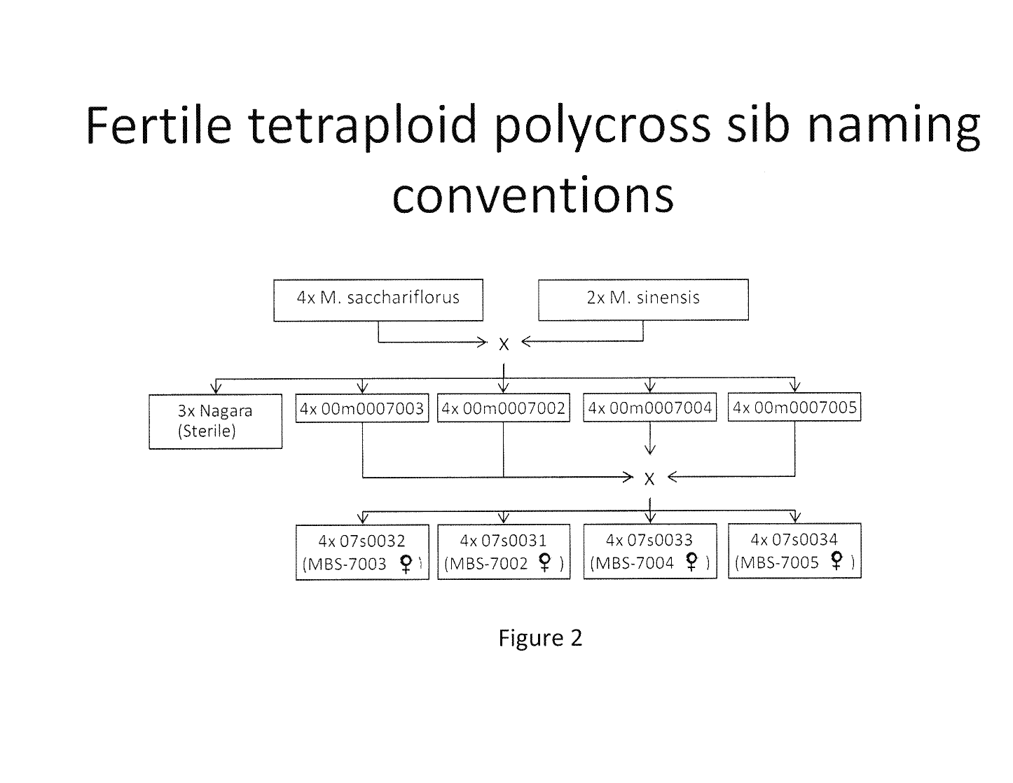

As discussed above and shown on FIG. 1, five clones were selected from the progeny resulting from crossing 4×M. sacchariflorus×2×M. sinensis. One selection is the 3× sterile clone, designated ‘MBS 7001’ (aka ‘Nagara’). The four other selections are the fertile tetraploid sibs designated 00 m0007002 (aka ‘MBS 7002’ or ‘Lake Erie’), 00 m000703 (aka ‘MBS 7003’ or ‘Columbia’), 00 m0007004 (aka ‘MBS 1001’) and 00 m0007005 (aka ‘MBS 1002’). See top half of FIG. 2 for a schematic representation of this process.

The fertile tetraploid (4x or 4n) sib varieties ‘MBS 7002, ‘MBS 7003,’ ‘MBS 1001’ and ‘MBS 1002’ were propagated clonally for use as parents in seed production. Each one of the fertile tetraploid sibs was used as a female plant and crossed to the other three fertile tetraploid sibs to produce the following four fertile tetraploid sib polycross families: 07s0031, with ‘MBS 7002’ used as the female parent (i.e., MBS 7002 [symbol] ×(MBS 7003 [symbol] , MBS 7004 [symbol] , MBS 7005 [symbol] ); 07s0032, with ‘MBS 7003’ used as the female parent (i.e., MBS 7003 [symbol] ×(MBS 7002 [symbol] , MBS 7004 [symbol] , MBS 7005 [symbol] ); 07s0033, with ‘MBS 7004’ used as the female parent (i.e., MBS 7004 [symbol] ×(MBS 7002 [symbol] , MBS 7003 [symbol] , MBS 7005 [symbol] ); and 07s0034, with ‘MBS 7005’ used as the female parent (i.e., MBS 7005 [symbol] ×(MBS 7002 [symbol] , MBS 7003 [symbol] , MBS 7004 [symbol] ). See bottom half of FIG. 2 for a schematic representation of this process. These polycross progeny are appropriate breeding proxies for the individual fertile tetraploid sib parents, each of which are self-incompatible.

The resultant seeds of the fertile tetraploid sib polycross families were germinated in flats or pots and transferred to the field for performance testing in replicated trials.

Controls included M.×giganteus (cv. ‘Illinois’) and the switchgrass varieties ‘Alamo’ and ‘Kanlow.’

A randomized complete block design was used. Plots consist of 4 rows of 8 plants on 0.75 m centers (3.0 m×6 m). There are 3 replications per location. To facilitate mechanical harvest, there was a 2.5 m alleys between plots. An additional border row of Miscanthus was planted on the outer two edges of the experiment to mitigate edge effects.

Seedlings in containers or plug cell trays were transplanted to the field (by hand, or mechanically if equipment is available) after the average historical date of potential freezing temperatures had occurred and 10 cm soil depth temperatures before 7:30 a.m. had increased to greater than 10° C.

Plants that died within the first 8 weeks from initial field-planting were replaced on a weekly basis. Missing/dead plants in the middle two rows were replaced with plants that were originally placed in the same plot's border rows. Missing plants in the border rows were replaced with additional seedlings.

At planting, 451b of N/acre was applied and, if needed, 10-151b P/acre; an additional N application of 451b/acre was be made 3 months after planting.

The planting year (year 1) was an establishment year so data collection in year 1 was limited. Yield and individual plant data were taken in year 2 and will be taken again in years 3-4. For yield data, the middle two rows of each plot are harvested. For individual plant data, all plants in each plot are measured.

Individual plant data was or will be collected according to the following instructions:
Culm Length (CmL)—Measure (cm) the plant's tallest culm, from the soil surface to the junction of the leaf blade and the sheath (the collar region) of the topmost leaf. If no flowering tillers are present, measure the tallest tiller to the topmost visible collar region. Take measurements during the last week of October.
Flowering Scale (FS)—Score fortnightly starting the 2^{nd} week of July through the end of October. Scale:

| | |
|---|---|
| 0 | not flowering |
| 5 | 1-4 flowering panicles |
| 9 | ≧5 flowering panicles. |

Percentage Flowering & Post Flowering Tillers (FPFT)—Indicate percentage of flowering and post-flowering tillers relative to the total number of tillers. Score during the 3^{rd} week of November. Scale:

| | |
|---|---|
| 1 | low (0-25%) |
| 3 | medium (26-50%) |
| 5 | high (51-75%) |
| 7 | very high (76-100%). |

Basal Circumference (BCirc)—Measure the uncompressed circumference (cm) of the plant at the soil surface. Use a digital tape measure. Take measurements during the 3^{rd} week of October.
Compressed Plant Circumference (CCirc)—Compress the tillers at 1 m above the soil surface such that adjacent tillers are in contact and the space is filled by tillers without air gaps. Measure the circumference (cm) of the compressed tillers. Use constant torque clamp and digital tape measure. Take measurements during the 3rd week of October.
Culm Diameter (CmD)—Measure the internode diameter (mm) of 1 typical mature culm at 1 m from the soil surface. Use digital calipers provided by Mendel. Take measurements during the 3^{rd} week of October.
Fall Leaf Senescence (Sen)—Evaluate the percentage of green leaves. Score fortnightly from the 2^{nd} week of September through the end of November. Scale:

| | | |
|---|---|---|
| 1 | 0-25% | green |
| 3 | 26-50% | green |
| 5 | 51-75% | green |
| 7 | 76-100% | green. |

Spring Re-growth Time (SRT)—Record the date on which at least one tiller is >50 cm tall. Walk the field with a measuring stick marked at 50 cm. Record at fortnightly intervals starting approximately mid March (depending on location) through the end of May (or until all living plants have reached 50 cm).
Survival (Surv)—Record if the plant is dead or alive. Score initially during the 1^{st} week of September of the establishment year, then each subsequent year during the 1^{St} week of May. Scale:

| | |
|---|---|
| 0 | dead |
| 1 | alive. |

Leaf Rolling (LRL)—Scale:

| | |
|---|---|
| 0 | Leaves healthy |
| 1 | Leaves start to fold (shallow V-shape) |
| 3 | Leaves folding (deep V-shape) |
| 5 | Leaves fully cupped (U-shape) |
| 7 | Leaf margins touching (O-shape) |
| 9 | Leaves tightly rolled. |

Leaf Drying (LDR)—Scale:

| | |
|---|---|
| 0 | No symptoms |
| 1 | Slight tip drying |
| 3 | Tip drying extending up to ¼ length in most leaves |
| 5 | ¼^{th} to ½ of all leaves fully dried |
| 7 | More than ⅔ of all leaves fully dried |
| 9 | All plants apparently dead or dormant. |

Whole plot data was and will be collected according to the following instructions:

Biomass Yield (Yld)—Make single annual harvests during late autumn through early winter, once tiller initiation has ceased and leaves are no longer green in all of the Miscanthus entries. Prior to harvest, trim blocks to a uniform length of 6 m. Plot harvest size will be 1.5 m (2 center rows) by 6 m at a 10 cm stubble height. Record wet weights for each plot, and a subsample of each plot followed by drying and weighing the subsamples in order to determine percent moisture content. If electronic equipment for estimating percent moisture content of the main harvest is available, then subsampling will only be needed for quality and nutrient composition tests.
Lodging (Lg)—Record the % of plants that lodged during the last week of October.
Tiller Density (TD)—Count the number of tillers within a 50 cm×50 cm square (0.25 square meter) in the central part of the plot—year 3 (only if plants have grown together such that single plant measures are no longer possible) and year 4. Take data after winter harvest & before spring growth initiation.
Soil Samples—For select plots, take immediately after planting, then once per year on the anniversary of the first measurement for N, P, K, soil carbon and pH. Use a 2-5 cm diameter soil core sampler or auger to collect to a depth of 1 m, at the mid-point between plants in a row, to minimize disruption of roots and to avoid soil compacted by machinery. Collect 3 randomized sub-samples per plot, and pool into a single sample per plot.
Whole trial data was or will be collected according to the following instructions:
Volunteering (Vol)—Scout for volunteer seedlings in and around the trial. Conduct a search during mid summer and again in autumn, when the distinctive inflorescence of Miscanthus will be visible.

The yield potential for the four fertile tetraploid sib polycross families and M×g variety ‘Illinois’ at six U.S. locations were determined in the second year of growth. Biomass yield values shown in dry Tonnes per U.S. acre (dT (U.S.)/acre) in Table 1, below. Harvests in Champaign and New Castle were by hand and early. Davis, Calif. location dropped in over-location averages (last column) because not all entries were present. As expected, the shorter and earlier 07s0032 yielded the least.

TABLE 1
Yield potential in year 2 (dT (U.S.)/acre) for fertile tetraploid sib polycross families.

| | | Providence | Jerseyville, | Champaign, | | | Over all locations, |
|---|---|---|---|---|---|---|---|
| Entry | Starkville, MS | Forge, VA | IL | IL | Leland, MS | New Castle, KY | excluding Davis |
| 07s0031 (MBS-7002) | 4.4 | 4.8 | 3.8 | 7.1 | 10.3 | 11.2 | 7.1 |
| 07s0032 (MBS-7003) | 5.1 | 4.6 | 6.4 | 7.1 | 7.1 | 8.2 | 6.4 |
| 07s0033 (MBS-7004) | 4.0 | 4.7 | 4.7 | 8.6 | 9.0 | 10.8 | 7.3 |
| 07s0034 (MBS-7005) | 3.4 | 5.1 | 4.4 | 9.2 | 9.8 | 11.0 | 7.3 |
| Mxg ‘Illinois’ | 4.3 | 5.3 | 6.1 | 12.3 | 8.8 | 13.1 | 8.2 |
| Site avg | 4.2 | 4.9 | 5.2 | 8.9 | 9.0 | 10.9 | 7.3 |
| Harvest Date: | 24-Feb | 26-Feb | 04-Feb | 23-Nov | 27-Jan | 20-Nov | |

The height in centimeters for the four fertile tetraploid sib polycross families, M×g variety ‘Illinois,’ and the switchgrass varieties ‘Alamo’ and ‘Kanlow’ were determined at 9 U.S. locations. Data is provided in Table 2.

TABLE 2
Height in cm at the end of October in year 2 for fertile tetraploid sib polycross families

| | | | | | | | | New | | Over all |
|---|---|---|---|---|---|---|---|---|---|---|
| | | Starkville, | Leland, | Auburn, | Jerseyville, | Davis, | Champaign, | Castle, | Providence | locations, excluding |
| Entry | Ames, IA | MS | MS | AL | IL | CA | IL | KY | Forge, VA | Ames & Davis |
| 07s0031 (MBS-7002) | | 201 | 233 | 239 | 248 | | 255 | 261 | 268 | 245 |
| 07s0032 (MBS-7003) | | 189 | 190 | 207 | 203 | | 218 | 218 | 212 | 207 |
| 07s0033 (MBS-7004) | 199 | 218 | 221 | 235 | 228 | 242 | 268 | 261 | 261 | 244 |
| 07s0034 (MBS-7005) | | 182 | 204 | 242 | 224 | | 263 | 234 | 246 | 231 |
| Alamo switchgrass | 145 | 207 | 213 | 214 | 185 | | 226 | | | 208 |
| Kanlow switchgrass | 164 | 210 | 190 | 210 | 201 | | 193 | | | 203 |
| Mxg ‘Illinois’ | 150 | 214 | 172 | 193 | 283 | 226 | 291 | 270 | 283 | 254 |
| Site avg | 164 | 203 | 207 | 220 | 225 | 234 | 247 | 250 | 254 | 230 |

The average yield of all four fertile tetraploid sib polycross families was determined for 8 locations and compared to the average yields of M×g variety ‘Illinois,’ and the switchgrass varieties ‘Alamo’ and ‘Kanlow’ at those same locations. See, Table 3, below. As an example of the method used to calculate the average yield for the fertile tetraploid sib polycross at any one location, the 4.2 dT (U.S.)/acre shown in Table 3 for Starkville, Miss., is the average of the four yields for each of the four fertile tetraploid sib polycross families shown for the same location in Table 1 (i.e., 4.4+5.1+4.0+3.4÷4=4.225≈4.2). Harvests in Davis, Champaign and New Castle were by hand and early. The fertile tetraploid polycross sibs, M.×giganteus ‘Illinois’ and Switchgrass yielded similarly overall in year-2.

TABLE 3
Yield potential in year 2 (dT (U.S.)/acre), for groups of key entry types

| | Auburn, | Starkville, | Jerseyville, | Providence | | | | | |
|---|---|---|---|---|---|---|---|---|---|
| Group | AL | MS | IL | Forge, VA | Davis, CA | Leland, MS | Champaign, IL | New Castle, KY | Over all locations |
| Mxg ‘Illinois’ | 2.1 | 4.3 | 6.1 | 5.3 | 4.5 | 8.8 | 12.3 | 13.1 | 6.4 |
| Fertile tetraploid | 4.2 | 4.2 | 4.9 | 4.8 | 8.3 | 9.1 | 8.0 | 10.3 | 6.8 |
| sib polycross* | | | | | | | | | |
| Switchgrass** | 3.8 | 5.1 | 5.1 | 6.3 | 9.9 | 7.5 | 8.9 | 9.1 | 7.1 |
| Site avg | 3.8 | 4.5 | 5.2 | 5.3 | 7.5 | 8.5 | 8.9 | 10.3 | 7.0 |

*Mean of all four fertile tetraploid polycross sib families
**Mean of cultivars Alamo and Kanlow

Similarly, the average stem diameter, spring regrowth time and fall dormancy time of the four fertile tetraploid sib polycross families was determined and compared to the average values for the same traits of M×g variety ‘Illinois,’ and the two switchgrass varieties ‘Alamo’ and ‘Kanlow.’ See, Table 4, below.

TABLE 4
Stem diameter, spring regrowth time & fall dormancy time
for genetic groups in year 2 (Locations: Starkville, MS, Leland,
MS, Auburn, AL, Jerseyville, IL, and Champaign, IL.)

| | | Spring | Fall |
|---|---|---|---|
| | Stem diameter | regrowth | dormancy |
| Group | (mm) | time | time |
| Max ‘Illinois’ | 6.9 | 1-May | 27-Oct |
| Fertile tetraploid sib polycross* | 7.0 | 25-Apr | 6-Nov |
| Switchgrass** | 5.6 | 29-Apr | 22-Oct |

*Mean of all four fertile tetraploid polycross sib families
**Mean of cultivars Alamo and Kanlow

M×g ‘Illinois’ and the fertile tetraploid sib polycross had thick stems of about the same size. switchgrass had the thinnest stems, which were also hollow, unlike the stems of the Miscanthus germplasms. The thin hollow stems likely contributed to lodging of the switchgrass. The better stem structure of the fertile tetraploid sib polycross suggests that gains in height can be made without much increased risk of lodging. Spring regrowth time was similar for all entries. Fertile tetraploid lines went dormant about one week later than M×g ‘Illinois’ and about two weeks later than switchgrass cultivars.

### Example IX

### Biomass Yield Predictions from the 4-Combination F1 Bulk Testing Results

The inventors of the present invention believe that the 4-way cross is representative of all the other parent pairings (i.e, 2-way and 3-way crosses) in terms of increased biomass compared to sterile Miscanthus varieties, such as M×g.

For example, the inventors expect to find that biomass from the 4-way cross, which includes

one parent that is earlier flowering than other parents (i.e., MBS 7003), would be, if anything, at the lower end of what would be expected for the 2-way combinations or the 3-way combinations without this early-flowering parent. This is because later flowering leads to increased height and vegetative plant biomass. Thus, based on the data provided herein, it is predicted that the yield data should be even better for plants grown from seed resulting from the di- or tri-parental crosses that do not include the earlier-flowering ‘MBS 7003’ used in the 4-parental cross which was used to obtain the data presented in Example VIII.

Early flowering is a key impediment to realizing the full biomass yield potential of Miscanthus, especially in the southern U.S. In sugarcane, flowering alone reduces yields by enough to wipe out profit margins in some years (Julien and Soopramanien (1976) Rev Agric Suer Ile Maurice 55:151-158; Long (1976) Proc South Afr Sugar Technol Assoc 50:78-81; Julien et al. (1978) Proc Int Soc Sugar Cane Technol 16:1771-1789; Heinz (1987) Sugarcane improvement through breeding. Elsevier, Amsterdam), and increases disease susceptibility. Though some M. sacchariflorus genotypes (especially from Japan) have a short-day response that confers autumn flowering, most M. sinensis genotypes flower several months before cool temperatures limit growth. Among 6 genes that control flowering of sorghum (Quinby (1966) Crop Science 6:516-518; Rooney and Aydin (1999) Crop Science 39:397-400), one has been cloned (Childs et al. (1997) Plant Physiology 113:611-619), the locations of three more are published (Lin et al. (1995) Genetics 141:391-411; Paterson et al. (1995) Science 269:1714-1718; Ulanch et al. (1996) Plant Physiology 111:709), and additional QTLs are known in sugarcane (Ming et al. (2002) Genome 45:794-803). QTL DTH8 (QTL for days to heading on chromosome 8) in rice (Oryza sativa) plays an important role in the signal network of photoperiodic flowering as a novel suppressor as well as in the regulation of plant height and yield potential (Wei et al. (2010) Plant Physiology 153:1747-1758).

As a general rule, for all grasses such as switchgrass, Miscanthus, sorghum, tropical maize, energycane, the switch from a vegetative growth phase to a flowering growth phase ends any significant accumulation of above-ground (i.e., shoot or top) vegetative biomass. Below-ground growth may continue to accumulate, principally in the rhizomes, but this growth is not direct contributor to biomass yield. From that point on, additional biomass is associated with flowering structures and seed only. For a crop like Miscanthus with very small and light seed, the proportion of biomass associated with flowering structures and seed is very small compared to vegetative biomass.

Since the present invention is mostly focused on overall biomass yield, not grain yield, the later the flowering time, the more time is allowed for vegetative biomass to accumulate. Thus, we anticipate that unless there is some unusual interaction between the genes in the four different parents (which we don't expect to find given the common parentage of these 4 parents) that the average flowering time of the product from the 4-way cross—for which we have yield data—will be slightly earlier than the flowering time of any other combination lacking a contribution of the

early-flowering parent. The range of variation in flowering time for the 4-way product is a fairly normal distribution, consistent with our interpretation.

The present invention is not limited by the specific embodiments described herein. The invention now being fully described, it will be apparent to one of ordinary skill in the art that many changes and modifications can be made thereto without departing from the spirit or scope of the present invention. Modifications that become apparent from the foregoing description and any accompanying figures fall within the scope of the present invention, or claims that may derive from the present invention.

Unless defined otherwise, all technical and scientific tennis herein have the same meaning as commonly understood by one of ordinary skill in the art to which this invention belongs. Although any methods and materials, similar or equivalent to those described herein, can be used in the practice or testing of the present invention, the preferred methods and materials are described herein. All publications, patents, and patent publications cited are incorporated by reference herein in their entirety for all purposes.

The publications discussed herein are provided solely for their disclosure prior to the filing date of the present application. Nothing herein is to be construed as an admission that the present invention is not entitled to antedate such publication by virtue of prior invention.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1. A fertile tetraploid Miscanthus variety which produces a biomass yield at least 90% of the biomass yield produced by Miscanthus×giganteus ‘Illinois’ when grown under substantially the same environmental conditions.

2. The fertile tetraploid Miscanthus variety of claim 1, wherein the fertile tetraploid Miscanthus variety has an average stem diameter at least 90% as large as the stem diameter of Miscanthus×giganteus ‘Illinois’ when grown under substantially the same environmental conditions.

3. The fertile tetraploid Miscanthus variety of claim 1, wherein the fertile tetraploid Miscanthus variety produces a biomass yield at least 90% of the biomass yield produced by Miscanthus×giganteus ‘Illinois’ and an average stem diameter at least 95% as large as the stem diameter produced by Miscanthus×giganteus ‘Illinois’ when grown under substantially the same environmental conditions.

4. The fertile tetraploid Miscanthus variety of claim 1, wherein the variety produces a biomass yield at least 100% of the biomass yield produced by Miscanthus×giganteus ‘Illinois.’

5. The fertile tetraploid Miscanthus variety of claim 1, wherein the fertile tetraploid Miscanthus variety produces a biomass yield at least 105% of the biomass yield produced by Miscanthus×giganteus ‘Illinois.’

6. The fertile tetraploid Miscanthus variety of claim 1, wherein the fertile tetraploid Miscanthus variety produces an average stem diameter at least 100% of the average stem diameter of Miscanthus×giganteus ‘Illinois.’

7. The fertile tetraploid Miscanthus variety of claim 1, wherein the fertile tetraploid Miscanthus variety produces an average stem diameter at least 105% of the average stem diameter of Miscanthus×giganteus ‘Illinois.’

8. The fertile tetraploid Miscanthus variety of claim 1, wherein the fertile tetraploid Miscanthus variety comprises germplasm from one or more varieties selected from the group consisting of ‘MBS 7002,’ ‘MBS 7003,’ ‘MBS 1001,’ and ‘MBS 1002.’

9. The fertile tetraploid Miscanthus variety of claim 8, wherein the fertile tetraploid Miscanthus variety is incorporated into feedstock for biofuel production, and said feedstock comprises plant biomass produced by a Miscanthus variety selected from the group consisting of ‘MBS 7002,’ ‘MBS 7003,’ ‘MBS 1001,’ and ‘MBS 1002.’

10. A Miscanthus hybrid, synthetic or open pollinated population wherein said hybrid, synthetic or open pollinated population comprises germplasm from one or more Miscanthus varieties selected from the group of varieties consisting of ‘MBS 7002,’ ‘MBS 7003,’ ‘MBS 1001,’ and ‘MBS 1002.’

11. The Miscanthus hybrid, synthetic or open pollinated population of claim 10, wherein the Miscanthus hybrid, synthetic or open pollinated population comprises fertile tetraploid Miscanthus plants.

12. The Miscanthus hybrid, synthetic or open pollinated population of claim 10, wherein the Miscanthus hybrid, synthetic or open pollinated population is selected from the group consisting of ‘MBS 7002’×‘MBS 7003’; ‘MBS 7002’×‘MBS 1001’; ‘MBS 7002’×“MBS 1002’; ‘MBS 7003’×‘MBS 1001’; ‘MBS 7003’×‘MBS 1002’; and ‘MBS 1001’×‘MBS 1002.’

13. The Miscanthus hybrid, synthetic or open pollinated population of claim 10, wherein the Miscanthus hybrid, synthetic or open pollinated population is selected from the group consisting of ‘MBS 7002’×‘MBS 7003’×‘MBS 1001’; ‘MBS 7002’×‘MBS 7003’×‘MBS 1002’; ‘MBS 7002’×‘MBS 1001’×“MBS 1002’ and ‘MBS 7003’×‘MBS 1001’×‘MBS 1002.’

14. The Miscanthus hybrid, synthetic or open pollinated population consisting of claim 10, wherein the Miscanthus hybrid, synthetic or open pollinated population consists of ‘MBS 7002’×‘MBS 7003’×‘MBS 1001’×‘MBS 1002.’

15. A method of producing a Miscanthus hybrid, synthetic or open pollinated population comprising crossing two or more fertile tetraploid Miscanthus varieties wherein at least one parent used to produce said hybrid, synthetic or open pollinated population is selected from the group of Miscanthus varieties consisting of ‘MBS 7002,’ ‘MBS 7003,’ ‘MBS 1001,’ and ‘MBS 1002.’

16. The method of claim 15, wherein at least two parents used to produce said hybrid, synthetic or open pollinated population are selected from the group of Miscanthus varieties consisting of ‘MBS 7002,’ ‘MBS 7003,’ ‘MBS 1001,’ and ‘MBS 1002.’

17. The method of claim 15, wherein at least three parents used to produce said hybrid, synthetic or open pollinated population are selected from the group of Miscanthus varieties consisting of ‘MBS 7002,’ ‘MBS 7003,’ ‘MBS 1001,’ and ‘MBS 1002.’

18. The method of claim 15, wherein the parents used to produce said hybrid, synthetic or open pollinated population comprise ‘MBS 7002,’ ‘MBS 7003,’ ‘MBS 1001,’ and ‘MBS 1002.’

19. (canceled)

20. The Miscanthus hybrid, synthetic or open pollinated population of claim 10, wherein at least one parent used to produce said hybrid, synthetic or open pollinated population is selected from the group of Miscanthus varieties consisting of ‘MBS 7002,’ ‘MBS 7003,’ ‘MBS 1001,’ and ‘MBS 1002.’
